(19) United States
(12) Patent Application Publication
(10) Pub. No.: US 2022/0364113 A1
(43) Pub. Date: Nov. 17, 2022

(54) HOST SYSTEMS COMPRISING INHIBITORS OF A GENE-EDITING PROTEIN FOR PRODUCTION OF VIRAL VECTORS

(71) Applicant: ASKLEPIOS BIOPHARMACEUTICAL, INC., Research Triangle Park, NC (US)

(72) Inventor: Lester SUAREZ, Research Triangle Park, NC (US)

(73) Assignee: ASKLEPIOS BIOPHARMACEUTICAL, INC., Research Triangle Park, NC (US)

(21) Appl. No.: 17/623,412

(22) PCT Filed: Jul. 2, 2020
(86) PCT No.: PCT/US2020/040677

§ 371 Date: Dec. 28, 2021

Related U.S. Application Data
(60) Provisional application No. 62/870,176, filed on Jul. 3, 2019.

(51) Int. Cl. C12N 15/86; C07K 16/40; C12N 15/10; G16B 35/20; C12N 9/22

(52) U.S. Cl. CPC C12N 15/86; C07K 16/40; C12N 15/1037; G16B 35/20; C12N 9/22; C12N 2750/14143; C12N 2750/14152; C12N 2310/20

(57) ABSTRACT

The present invention provides a method of manufacturing vectors containing a heterologous gene-editing protein comprising providing (a) transforming a host system with a nucleic acid cassette containing a promoter operably linked to a gene encoding a gene-editing protein, wherein the host system also contains a heterologous inhibitor for the gene-editing protein, (b) incubating the host system for a time sufficient for vector production and to release the recombinant vector, and (c) recovering the recombinant vector. Also provided herein are cell lines for expressing vectors containing a gene-editing protein with an inhibitor of the gene-editing protein to prevent leaky expression of the gene-editing protein comprising constitutive expression of an inhibitor of a gene-editing protein.

### CROSS-REFERENCE TO RELATED APPLICATIONS

This application claims benefit under 35 U.S.C. § 119(e) of the U.S. provisional application No. 62/870,176 filed Jul. 3, 2019, the contents of which are incorporated herein by reference in their entirety

### FIELD OF THE INVENTION

The present invention relates to methods and cell lines for generating viral vectors containing gene-editing systems.

### BACKGROUND OF THE INVENTION

Genome editing technology, such as the CRISPR/Cas system, represents a highly promising area to advance the future of therapy for inherited and acquired genetic alterations. CRISPR gene therapy has shown promising results for many diseases including, but not limited to, cancer, beta-thalassemia, hereditary forms of blindness, cystic fibrosis, HIV/AIDS infection, muscular dystrophy, and Huntington's disease. Engineered CRISPR systems contain two components necessary for Cas-based genome modification—the CRISPR-associated endonuclease (Cas protein) and a guide RNA (gRNA). For many applications, viral vectors, e.g., recombinant adeno-associated viral (rAAV) vectors, represent optimal vehicles to achieve high-efficiency delivery of the CRISPR-Cas system for gene-editing of a cell. However, unintentional expression of non-human proteins, such as the Cas protein, is undesirable in cells. Immunity to Cas9 has been found in humans and animal models, and there is growing concern that prolonged Cas expression in a human cell could provoke severe tissue damage (e.g., genotoxicity due to off-target nuclease activity) and elimination of the edited cell(s).

The Self-Inactivating/Deleting CRISPR System for viral vectors (e.g., rAAV) has been shown to dramatically reduce the level of Cas protein expression, while achieving high rates of on-target editing. This system functions as a negative feedback loop in which Cas cuts both the target genomic locus and its own coding construct, and thereby self-limits its own expression. Some self-inactivating AAV-CRISPR systems rely on efficient co-transduction with two different AAV vectors, but variations in co-delivery likely result in the differences observed in both editing efficiency and vector toxicity. In order to guarantee delivery of the Cas protein, on-target gRNA, and self-deleting gRNA to the cell at substantially the same time, others have attempted to utilize a single vector system. However, the development of a single vector system in which the self-deleting gRNA was co-expressed in cis with Cas has been unsuccessful, or very inefficient due to manufacturing issues. Indeed, the unintentional expression of gene editing proteins during manufacture of vector systems containing such proteins has reduced yield of such manufacturing systems. Described herein is novel method for efficient inactivation of a gene-editing system (for example, a Cas protein) in a viral vector.

### SUMMARY OF INVENTION

One aspect of the invention described herein provides a method of manufacturing vectors containing a heterologous gene-editing protein comprising, providing (a) transforming a host system with a nucleic acid cassette containing a promoter operably linked to a gene encoding a gene-editing protein, wherein the host system also contains a heterologous inhibitor for the gene-editing protein; (b) incubating the host system for a time sufficient for vector production and then releasing the recombinant vector; and (c) recovering the recombinant vector.

In various embodiments of any aspect, the host system is a host cell or a cell-free system.

In one embodiment of any aspect, the host cell is a mammalian cell or an insect cell.

In one embodiment in any aspect, the promoter is an inducible promoter. In one embodiment in any aspect, the promoter is a tissue-specific promoter.

In one embodiment of any aspect, the vector is a viral vector. In one embodiment of any aspect, the vector is a DNA or RNA virus. Exemplary viral vectors include, but are not limited to, an adeno associated virus (AAV), a lentivirus (LV), a herpes simplex virus (HSV), an adeno virus (AV), or a pox virus (PV).

In one embodiment of any aspect, the nucleic acid cassette is flanked by terminal repeats.

In one embodiment of any aspect, the viral vector is an AAV vector and the nucleic acid cassette is flanked by inverted terminal repeats (ITRs).

In one embodiment of any aspect, viral vector is an AV vector and the nucleic acid cassette is flanked by inverted terminal repeats (ITRs).

In one embodiment of any aspect, the viral vector is an LV vector and the nucleic acid cassette is flanked by long terminal repeats (LTRs).

In one embodiment of any aspect, the inhibitor of a gene-editing protein is an anti-CRISPR protein that is not native to the host cell, i.e., a heterologous inhibitor. Exemplary anti-CRISPR proteins include, but are not limited to, the proteins listed in Tables 1 and 2.

In one embodiment of any aspect, the heterologous inhibitor of a gene-editing protein is an antibody that binds to the HNH domain of a Cas protein. An antibody can be, for example, a single chain antibody, a fragment antigen-binding antibody, or an intrabody.

In one embodiment of any aspect, binding the HNH domain results in a conformational change to the structure of the Cas protein. In one embodiment of any aspect, binding the HNH domain inactivates the activity of the Cas protein. For example, at least 5%, 10%, 20%, 30%, 40%, 50%, 60%, 70%, 80%, 90%, 99% or more of the activity is inactivated.

In one embodiment of any aspect, a second nucleic acid cassette containing a promoter operably linked to a gene encoding the heterologous inhibitor of a gene-editing protein is administered to the host system prior to step (a) of claim 1, or co-administered with step (a) of claim 1.

In one embodiment of any aspect, the host system constitutively expresses the heterologous inhibitor of a gene-editing protein. In one embodiment of any aspect, the host system transiently expresses the heterologous inhibitor of a gene-editing protein.

In one embodiment of any aspect, the second nucleic acid cassette is administered by a plasmid, a virus, a liposome, a microcapsule, a non-viral vector, or as naked DNA.

In one embodiment of any aspect, the gene-editing protein is the CRISPR-Cas 9 gene-editing system.

In one embodiment of any aspect, the gene-editing protein is a Cas protein. Exemplary Cas proteins include Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c. In one embodiment of any aspect, the Cas protein is Cas9.

In one embodiment of any aspect, the Cas protein is a Cas9 variant selected from Staphylococcus aureus (SaCas9), Streptococcus thermophilus (StCas9), Neisseria meningitidis (NmCas9), Francisella novicida (FnCas9), and Campylobacter jejuni (CjCas9).

In one embodiment of any aspect, the Cas protein has been modified for gene-editing without double strand DNA breaks (such as CRISPRi or CRISPRa) and is selected from the group consisting of dCas, nCas, and Cas 13.

In one embodiment of any aspect, the Cas protein is codon optimized for expression in the eukaryotic cell.

A second aspect of the invention described herein provides a cell line for expressing vectors containing a gene-editing protein with an inhibitor of the gene-editing protein to prevent leaky expression of the gene-editing protein comprising constitutive expression of an inhibitor for the gene-editing protein. In one embodiment, the cell is a eukaryotic or prokaryotic cell.

In one embodiment of any aspect, the cell further expresses a gene-editing protein. In one embodiment of any aspect, the expression of the gene-editing protein is transient. In one embodiment of any aspect, the expression of the gene-editing protein is constitutive.

A third aspect of the invention described herein provides an antibody that binds to the HNH domain of a Cas protein.

A fourth aspect of the invention described herein provides a method of identifying a peptide capable of inhibiting a gene-editing protein comprising (a) immobilizing a gene-editing protein sequence in a well of a microtiter plate; (b) introducing the phage display library to the microtiter plate for a time sufficient to allow for binding of the phage to the gene-editing protein; (c) identifying any peptide bound to the gene-editing protein as a candidate peptide; and (d) testing/evaluating the candidate peptides identified in steps (a)-(c) through one or more in vitro assays for their ability to modulate the nuclease activity of a gene-editing protein.

In one embodiment of any aspect, the gene-editing protein sequence is a Cas protein sequence. In one embodiment of any aspect, the gene-editing protein sequence is a HNH domain sequence of a Cas protein.

In one embodiment of any aspect, phage display library is a phage display peptide library.

A fifth aspect of the invention described herein provides a method of identifying a peptide capable of inhibiting a gene-editing protein comprising (a) screening peptide libraries using in silico high throughput docking for candidate peptides that are selectively identified for their ability to target and disrupt the nuclease activity of a gene-editing protein; and (b) testing/evaluating the candidate peptides identified in step (a) through one or more in vitro assays for their ability to modulate the nuclease activity of a gene-editing protein.

A sixth aspect of the invention described herein provides a peptide that inhibits a gene-editing protein identified using any of the methods described herein.

In one embodiment in any aspect, the inhibitor of the gene-editing protein is any peptide that inhibits a gene-editing protein described herein.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, “gene-editing” refers to altering the DNA sequence of a cell, by way of non-limiting example, by expressing in the cell a protein, e.g., a gene-editing protein, that causes a mutation or alteration in the DNA of the cell. Alterations of the DNA can include, but are not limited to, nucleotide deletions, nucleotide substitutions, and nucleotide additions. As used herein, a “gene-editing protein” refers to a protein that can, either alone or in combination with one or more other molecules, alter the DNA sequence of a cell, by way of non-limiting example, a nuclease, a transcription activator-like effector nuclease (TALEN), a zinc-finger nuclease, a meganuclease, a nickase, a clustered regularly interspaced short palindromic repeat (CRISPR)-associated protein or a natural or engineered variant, family-member, orthologue, fragment or fusion construct thereof.

As used herein, “heterologous” refers to a polypeptide which is not ordinarily produced by the host cell, e.g., the cell expressing the heterologous polypeptide, but rather, is derived from an organism different from the host cell. For example, the heterologous inhibitor used herein is derived, for example, from a bacterial cell and expressed in, for example, an insect or mammalian cell.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the nuclease activity in a cell that does not express the heterologous inhibitor of a gene-editing protein) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. Where applicable, a decrease can be preferably down to a level accepted as within the range of normal for an individual without a given disorder.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physicochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. ligan-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a polypeptide), e.g., a heterologous inhibitor of a gene-editing protein, can be a functional fragment of one of the amino acid sequences described herein, or of any anti-CRISPR protein sequence. As used herein, a “functional fragment” is a fragment or segment of a peptide which retains at least 50% of the wildtype reference polypeptide's activity according to an assay known in the art or described below herein. For example, a functional fragment of a heterologous inhibitor of a gene-editing protein would retain at least 50% of its capacity to inhibit a gene-editing protein. One skilled in the art can assess the capacity to inhibit a gene-editing protein using standard techniques, for example those described herein below. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

As used herein, the term “DNA” is defined as deoxyribonucleic acid. The term “polynucleotide” is used herein interchangeably with “nucleic acid” to indicate a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, “a,” “an” or “the” can be singular or plural, depending on the context of such use. For example, “a cell” can mean a single cell or it can mean a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a composition of this invention, dose, time, temperature, and the like, is meant to encompass variations of ±20%, ±10%, ±5%, ±1%, ±0.5%, or even ±0.1% of the specified amount.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The term “consisting of” refers to compositions, methods, and respective components thereof as described herein, which are exclusive of any element not recited in that description of the embodiment.

### BRIEF DESCRIPTION OF THE DRAWINGS

FIGS. 1A-1E depicts a schematic of the production of self-inactivating AAV-CRISPR vector via transfection. (FIG. 1A) Cloning of the anti-CRISPR proteins (AcrIIA2, AcrIIA4 and/or AcrIIC1 into their helper plasmid. (FIG. 1B) Transfection of the plasmids. (FIG. 1C) Production in Cell Lines. (FIG. 1D) Purification Procedure. (FIG. 1E) Validation using Next Generation Sequencing (in order to sequence the DNA packaged inside the viral particles) and/or Western Blot analysis to detect the Cas9 protein expression.

FIG. 2 depicts a schematic construction of an anti-CRISPR eukaryotic vectors for a Self-Inactivating/Deleting AAV-CRISPR vector production. The anti-CRISPR proteins, AcrIIA2, AcrIIA4 and/or AcrIIC1, were inserted into the plasmid XX680 (Ad helper plasmid) at the AcII site, under control of the CMV promoter.

FIGS. 3A-3B depict a schematic representation of the CRISPR-Cas9-mediated genome editing. (FIG. 3A) The anti-CRISPR can block at distinct steps of Cas9-mediated target DNA cleavage: 1)—The assembly of single guide RNA (gRNA) with the Cas9 protein, 2)—Prevent DNA binding and/or 3)—Active site deactivation. (FIG. 3B) Schematic representation of recombinant adeno-associated virus (rAAV) transduction and a recombinant genome containing the elements for self-inactivating and therapeutic genome editing.

FIGS. 4A-4B depicts the production of rAAV vectors based on triple plasmid transfection. (FIG. 4A) A schematic drawing for the production of rAAV vectors based on the exclusive use of anti-CRISPR-Ad-Helper plasmid. (FIG. 4B) Production of rAAV vectors based on a novel technology that uses stable anti-CRISPR cell line.

FIGS. 5A-5C demonstrates the CRISPR/Cas9 cleavage activity by anti-CRISPR-Proteins. (FIG. 5A) 2A self-cleaving peptide-based multi-anti-CRISPR-Proteins expression system: A bi_cistronic expression cassette contains the ORFs for AcrIIA4 and AcrIIA2. (FIG. 5B) Plasmids structure. Ad-Helper plasmid, based on XX680, encoding adenoviral helper sequences; the anti-CRISPR-Ad-Helper plasmid, based on XX680, contains the anti-Self-Inactivating/Deleting CRISPR System [From FIG. 5A], and Self-inactivating AAV-CRISPR plasmid, a single vector system, with the self-deleting gRNA and the ORF for Cas9. (FIG. 5C) Inhibition of the Self-inactivating AAV-CRISPR System by the anti-CRISPR-Ad-Helper plasmid. Validation of target site cleavage by T7 Endonuclease I (T7E1) assay.

FIG. 6 depicts a schematic of an unrooted phylogenetic trees of Cas9 proteins. Cas9 orthologs targeted by Acr (Anti-CRISPR) proteins are indicated with circles at ends of branches.

FIG. 7 depicts three distinct modes of CRISPR-Cas12a inactivation. Models for AcrVA1, AcrVA4 and AcrVA5 inhibition of Cas12a are presented herein. Cas12a assembles with its crRNA to form a surveillance complex (top). In the absence of inhibitors, Cas12a recognizes a complementary target DNA activating the RuvC leading to target interference and immunity. Phage-encoded AcrVA1 associates with Cas12a, triggering crRNA spacer truncation, which prevents DNA binding. Phage-encoded AcrVA4 dimerizes Cas12a and blocks dsDNA binding. Phage-encoded AcrVA5 blocks Cas12a dsDNA binding via an unknown mechanism. See, e.g., Knott, G. J., et al. 2019. Nature Struct and Mol Biol.

FIG. 8 is a schematic depicting secondary structures of Cas9 from different bacteria. CTD is responsible for both the PAM recognition and the guide RNA repeat-anti-repeat heteroduplex binding. It displays a Cas9-specific fold and contains PAM-interacting (PI) sites required for PAM interrogation. The PI domain forms an elongated structure comprising seven α-helices (α46-α52), a three-stranded antiparallel β-sheet (β18-β20), a five-stranded antiparallel β-sheet (β21-β23, β26 and β27), and a two-stranded antiparallel β-sheet (β24 and β25). Target recognition strictly requires the presence of a short PAM flanking the target site. PI domain is positioned to recognize the PAM sequence on the non-complementary DNA strand.

### DETAILED DESCRIPTION OF THE INVENTION

The present invention provides a method of manufacturing vectors containing a heterologous gene-editing protein comprising (a) transforming a host system with a nucleic acid cassette containing a promoter operably linked to a gene encoding a gene-editing protein, wherein the host system also contains a heterologous inhibitor for the gene-editing protein; (b) incubating the host system for a time sufficient for vector production, and releasing the recombinant vector; and (c) recovering the recombinant vector.

Viral vector Production

Protocols for producing recombinant viral vectors and for using viral vectors for nucleic acid delivery can be found, e.g., in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997). Further, production of AAV vectors is further described, e.g., in U.S. Pat. No. 9,441,206, the contents of which is incorporated herein by reference in its entirety.

Nonlimiting examples of vectors employed in the methods of this invention include any nucleotide construct used to deliver nucleic acid into cells, e.g., a plasmid, a nonviral vector or a viral vector, such as a retroviral vector which can package a recombinant retroviral genome (see e.g., Pastan et al., Proc. Natl. Acad. Sci. U.S.A. 85:4486 (1988); Miller et al., Mol. Cell. Biol. 6:2895 (1986)). For example, the recombinant retrovirus can then be used to infect and thereby deliver an inhibitor of a gene-editing protein of the invention to the infected cells. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naldini et al., Science 272:263-267, 1996), pseudotyped retroviral vectors (Agrawal et al., Exper. Hematol. 24:738-747, 1996), and any other vector system now known or later identified. Also included are chimeric viral particles, which are well known in the art and which can comprise viral proteins and/or nucleic acids from two or more different viruses in any combination to produce a functional viral vector. Chimeric viral particles of this invention can also comprise amino acid and/or nucleotide sequence of non-viral origin (e.g., to facilitate targeting of vectors to specific cells or tissues and/or to induce a specific immune response). Incubation conditions (e.g., timing, climate, medium, etc.) for a given condition are known in the art and can be readily identified by a skilled practitioner.

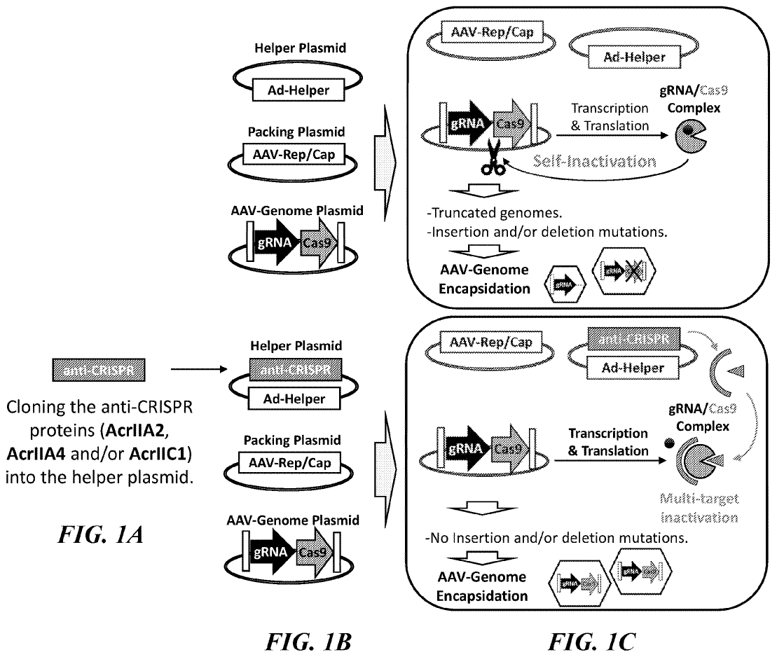

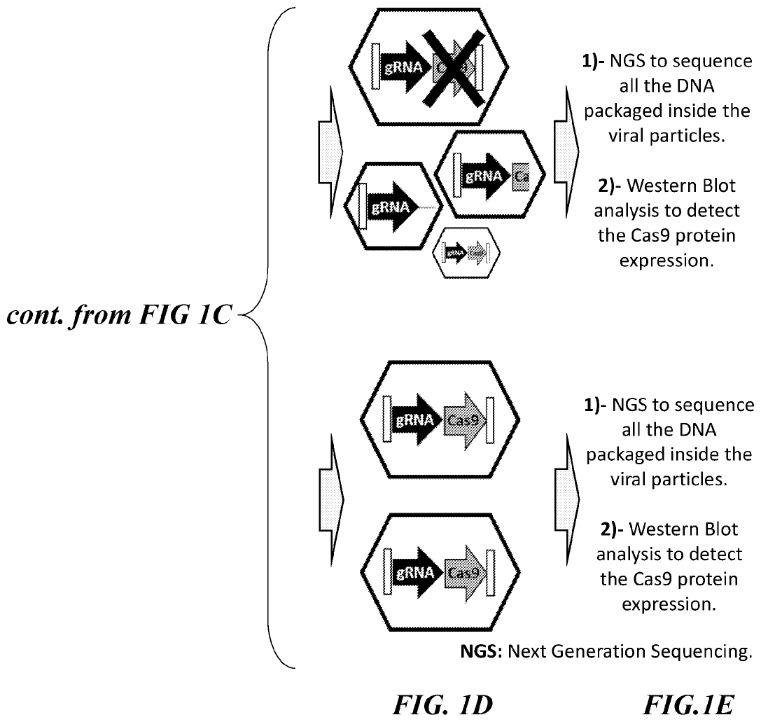

In one embodiment, the system described herein is for efficient production of a self-inactivating virus that can express both Cas9 and a self-deleting gRNA in one vector (see FIG. 1A-1E). Remarkably, it is found that this system inhibits broad spectrum of Cas orthologs, including Cas12a/Cpf1.

In one embodiment, the system described herein expresses the inactivating partner of the CRISPR system, known as anti-CRISPR proteins. These anti-CRISPR proteins derived from, for example, Bacteriophages, and include, but are not limited to, AcrIIC1, AcrIIA1, AcrIIA2, AcrIIA3, AcrIIA4. Anti-CRISPR proteins can include wild-type or mutant forms of these proteins, e.g., including functional fragments of anti-CRISPR.

Viral vectors produced in a host system can be released (i.e. set free from the cell that produced the vector) using any standard technique. For example, viral vectors can be released via mechanical methods, for example microfluidization, centrifugation, or sonication, or chemical methods, for example lysis buffers and detergents. Released viral vectors are then recovered (i.e., collected) and purified to obtain a pure population using standard methods in the art. For example, viral vectors can be recovered from a buffer they were released into via purification methods, including a clarification step using depth filtration or Tangential Flow Filtration (TFF). As described herein in the examples, viral vectors can be released from the cell via sonication and recovered via purification of clarified lysate using column chromatography.

In one embodiment, the vector can be, but is not limited to a nonviral vector or a viral vector. In one embodiment of any aspect, the vector is a DNA or RNA virus. Nonlimiting examples of a viral vector of this invention include an AAV vector, an adenovirus vector, a lentivirus vector, a retrovirus vector, a herpesvirus vector, an alphavirus vector, a poxvirus vector, a baculovirus vector, and a chimeric virus vector.

Any viral vector that is known in the art can be used in the present invention. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Peaenation mosaic virus group; Phycodnaviridae; Picornaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites.

Viral vectors produced by the method of the invention may comprise the genome, in part or entirety, of any naturally occurring and/or recombinant viral vector nucleotide sequence (e.g., AAV, AV, LV, etc.) or variant. Viral vector variants may have genomic sequences of significant homology at the nucleic acid and amino acid levels, produce viral vector which are generally physical and functional equivalents, replicate by similar mechanisms, and assemble by similar mechanisms.

Variant viral vector sequences can be used to produce viral vectors in the host system described herein. For example, or more sequences having at least about 70%, at least about 75%, at least about 80%, at least about 85%, at least about 90%, at least about 95%, at least about 99%, or more nucleotide and/or amino acid sequence identity (e.g., a sequence having about 75-99% nucleotide sequence identity) to a given vector (for example, AAV, AV, LV, etc.).

A host system will further be modified to include any necessary elements required to complement a given viral vector during its production using methods described herein. For example, in certain embodiment, the nucleic acid cassette is flanked by terminal repeat sequences. Complementary elements for a given viral vector are well known the art and a skilled practitioner would be capable of modifying the host system described herein accordingly. In one embodiment, for the production of rAAV vectors, the AAV host system will further comprise at least one of a recombinant AAV plasmid, a plasmid expressing Rep, a plasmid expressing Cap, and an adenovirus helper plasmid. Complementary elements for a given viral vector are well known the art and a skilled practitioner would be capable of modifying the viral expression system described herein accordingly.

For example, in certain embodiments, if the methods described herein are used to manufacture an AAV vector, the nucleic acid cassette (e.g., for expression of an anti-CRISPR protein) will be flanked by inverted terminal repeats (ITRs) in cis, which are necessary for integration of retroviral DNA. ITRs are typically 145 base pair symmetric sequences that flank the sequence intended to be expressed in the virus. The ITRs form a hairpin which contributes to self-priming that allows primase-independent synthesis of the second DNA strand. The ITRs are additionally required for both integration of the AAV DNA into the host cell genome and rescue from it, as well as for efficient encapsidation of the AAV DNA combined with generation of a fully assembled, deoxyribonuclease-resistant AAV particles.

A host system for manufacturing an AAV vector could further comprise Replication (Rep) genes and/or Capsid (Cap) genes in trans. On the left side of the AAV genome there are two promoters called p5 and p19, from which two overlapping messenger ribonucleic acids (mRNAs) of different length can be produced. Each of these contains an intron which can be either spliced out or not, resulting in four potential Rep genes; Rep78, Rep68, Rep52 and Rep40. Rep genes (specifically Rep 78 and Rep 68) bind the hairpin formed by the ITR in the self-priming act and cleave at the designated terminal resolution site, within the hairpin. They are necessary for the AAVS1-specific integration of the AAV genome. All four Rep proteins were shown to bind ATP and to possess helicase activity. The right side of a positive-sensed AAV genome encodes overlapping sequences of three capsid proteins, VP1, VP2 and VP3, which start from one promoter, designated p40. The cap gene produces an additional, non-structural protein called the Assembly-Activating Protein (AAP). This protein is produced from ORF2 and is essential for the capsid-assembly process. Necessary elements for manufacturing AAV vectors are known in the art, and can further be reviewed, e.g., in U.S. Pat. Nos. 5,478,745A; 5,622,856A; 5,658,776A; 6,440,742B1; 6,632,670B1; 6,156,303A; 8,007,780B2; 6,521,225B1; 7,629,322B2; 6,943,019B2; 5,872,005A; and U.S. Patent Application Numbers US 2017/0130245; US20050266567A1; US20050287122A1; the contents of each are incorporated herein by reference in their entireties.

In one embodiment, the host cells for producing an AAV vector are cultured in suspension. In another embodiment, the cells are cultured in animal component-free conditions. The animal component-free medium can be any animal component-free medium (e.g., serum-free medium) compatible with a given cell line, for example, HEK293 cells. Any cell line known in the art to be capable of propagating an AAV vector can be used for AAV production using methods described herein. Exemplary cell lines that can be used to generate an AAV vector include, without limitation, HEK293, CHO, Cos-7, and NSO.

In one embodiment, a host cells for producing an AAV vector stably expresses any of the components required for AAV vector production, e.g., Rep, Cap, VP1, etc. In one embodiment, a cell line for producing an AAV vector transiently expresses any of the components required for AAV vector production, e.g., Rep, Cap, VP1, etc.

In the event that the cells of the host system for producing an AAV vector does not stably or transiently express rep or cap, and these sequences are to be provided to the AAV host system, e.g., via an expression plasmid. AAV rep and cap sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the Ela or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., arc stably integrated into the cell as extra-chromosomal elements, designated as an “EBV based nuclear episome,” see Margolski, Curr. Top. Microbial. Immun. 158:67 (1992)).

Typically, the AAV rep/cap sequences will not be flanked by the TRs, to prevent rescue and/or packaging maintain of these sequences.

A host system for manufacturing a lentivirus using methods described herein would further comprise long terminal repeats (LTRs) flanking the nucleic acid cassette. LTRs are identical sequences of DNA that repeat hundreds or thousands of times at either end of retrotransposons or proviral DNA formed by reverse transcription of retroviral RNA. The LTRs mediate integration of the retroviral DNA via an LTR specific integrase the host chromosome. LTRs for manufacturing lentiviral vectors are further described, e.g., in U.S. Pat. No. 7,083,981B2; U.S. Pat. No. 6,207,455B1; U.S. Pat. No. 6,555,107B2; U.S. Pat. No. 8,349,606B2; U.S. Pat. No. 7,262,049B2; and U.S. Patent Application Numbers US20070025970A1; US20170067079A1; US20110028694A1; the contents of each are incorporated herein by reference in their entireties.

A host system for manufacturing an adenovirus using methods described herein would further comprise identical Inverted Terminal Repeats (ITR) of approximately 90-140 base pairs (exact length depending on the serotype) flanking the nucleic acid cassette. The viral origins of replication are within the ITRs exactly at the genome ends. The adenovirus genome is a linear double-stranded DNA molecule of approximately 36000 base pairs. Often, adenoviral vectors used in gene therapy have a deletion in the E1 region, where novel genetic information can be introduced; the E1 deletion renders the recombinant virus replication defective. ITRs and methods for manufacturing adenovirus vectors are further described, e.g., in U.S. Pat. No. 7,510,875B2; U.S. Pat. No. 7,820,440B2; U.S. Pat. No. 7,749,493B2; U.S. Pat. No. 7,820,440B2; U.S. Ser. No. 10/041,049B2; International Patent Application Numbers WO2000070071A1; and U.S. Patent Application Numbers WO2000070071A1; US20030022356A1; US20080050770A1 the contents of each are incorporated herein by reference in their entireties.

In one embodiment, the host system is a host cell. For example, the host cell is a virus, a mammalian cell or an insect cell. Exemplary insect cells include but are not limited to Sf9, Sf21, Hi-5, and S2 insect cell lines. For example, a host system for manufacturing an AAV vector could further comprise a baculovirus expression system, for example, if the host system is an insect cell. The baculovirus expression system is designed for efficient large-scale viral production and expression of recombinant proteins from baculovirus-infected insect cells. Baculovirus expression systems are further described in, e.g., U.S. Pat. No. 6,919,085B2; U.S. Pat. No. 6,225,060B1; U.S. Pat. No. 5,194,376A; the contents of each are incorporated herein by reference in their entireties.

In another embodiment, the host system is a cell-free system. Cell-free systems for viral vector production are further described in, for example, Cerqueira A., et al. Journal of Virology, 2016; Sheng J., et al. The Royal Society of Chemistry, 2017; and Svitkin Y. V., and Sonenberg N. Journal of Virology, 2003; the contents of which are incorporated herein by reference in their entireties.

In one embodiment, the host system is a cell free method of synthesis. For example, the vectors and/or cassettes can be synthesized and assembled in an in vitro system. One can prepare cassettes that will express the necessary enzymatic protein, e.g., for lentivirus, pol; for AAV, Rep. Other cassettes can be assembled that will express the necessary structural proteins, e.g., for lentivirus, gag and env; for AAV, the cap gene that expresses VP1, VP2, and VP3. Another vector will be synthesized having a gene operably linked to the desired transgene, which is ultimately flanked between packaging sequences, such as a LTR or an ITR. Various methods to accomplish this are known in the art.

### Anti-CRISPR Proteins

In one embodiment, the inhibitor of a gene-editing protein is an anti-CRISPR protein. CRISPR proteins provide bacteria with RNA-based adaptive immunity against phage infection. To counteract this defense mechanism, phages evolved anti-CRISPR (Acr) proteins that inactivate the CRISPR-Cas systems. Several Acr proteins have been identified; Acr proteins encoded by Listeria monocytogenes prophages are the most prevalent among the Acr proteins targeting type II-A CRISPR-Cas systems. an anti-CRISPR protein is characterized by its ability to inactivate or reduce the nuclease activity of a gene-editing protein, for example, by at least 50%, at least 60%, at least 70%, at least 80%, at least 90%, at least 95%, at least 99%, or more. An anti-CRISPR protein can inactivate or reduce the nuclease activity via various mechanisms, for example, by inducing an inhibitory conformational change or a post-translation modification, e.g., acetylation, to the gene-editing protein. Methods and assays for determining if an anti-CRISPR protein inactivates or reduces the nuclease activity of a gene-editing protein are further described herein.

Arc proteins are further described in, for example, in Maxwell, K L. 2017. “The Anti-CRISPR Story: A Battle for Survival,” Molecular Cell.; Rauch, B L, et al. 2017. “Inhibition of CRISPR-Cas9 with Bacteriophage Proteins,” Cell.; Pawluk, A, et al. 2016. “Naturally Occurring Off-Switches for CRISPR-Cas9,” Cell.; Cross R. 2018. “New CRISPR inhibitors found with help from U.S. Defense Department funding,” Chemical and Engineering News; Lee, J, et al. 2018. “Potent Cas9 Inhibition in Bacterial and Human Cells by AcrIIC4 and AcrIIC5 Anti-CRISPR Proteins.” MBio; Harrington, L B, et al. 2017. “A Broad-Spectrum Inhibitor of CRISPR-Cas9.” Cell.; Knott, G. J., et al. 2019. “A Broad-Spectrum Inhibitor of CRISPR-Cas12a.” Nature Struct and Mol Biol.; Shi, J, et al. 2017. “Disabling Cas9 by an anti-CRISPR DNA mimic.” Science Advances.; Li, C, et al. 2018. “HDAd5/35++ Adenovirus Vector Expressing Anti-CRISPR Peptides Decreases CRISPR/Cas9 Toxicity in Human Hematopoietic Stem Cells.” Mol Ther Methods Clin Dev.; Bubeck, F, et al. 2018. “Engineered anti-CRISPR proteins for optogenetic control of CRISPR-Cas9.” Nature Methods.; Dong, L., et al. 2019. “An anti-CRISPR protein disables type V Cas12a by acetylation.” Nature Struct and Mol Biol.; and Marino, N D., et al. 2019. “Discovery of widespread Type I and Type V CRISPR-Cas inhibitors.” Science; the contents of which are incorporated herein by reference in their entireties.

Table 1 is a non-limiting list of known anti-CRISPR proteins. Anti-CRISPR proteins in Table 1 can be further reviewed in, e.g., Zhang, F., et al. Animal Models and Exp. Medicine, 2019. An anti-CRISPR protein of the present invention can be any variant, orthologs, paralog, homolog, or chain of the Anti-CRISPR proteins listed in Table 1.

TABLE 1

| Anti- | | Length | CRISPR-Cas system |
|---|---|---|---|
| CRISPR | Origin | (a. acids) | inhibited |
| AcrIC1 | Moraxella bovoculi prophage | 190 | I-C (Pae) |
| AcrID1 | Sulfolobus islandicus rudivirus 3 | 104 | I-D (Sis) |
| AcrIE1 | Pseudomonas aeruginosa phage | 100 | I-E (Pae) |
| | JBD5 | | |
| AcrIE2 | P aeruginosa phage JBD88a | 84 | I-E (Pae) |
| AcrIE3 | P aeruginosa phage DMS3 | 68 | I-E (Pae) |
| AcrIE4 | P aeruginosa phage D3112 | 52 | I-E (Pae) |
| AcrIE4-F7 | Pseudomonas citronellolis | 119 | I-E/I-F (Pae) |
| | prophage | | |
| AcrIE5 | Pseudomonas otitidis prophage | 65 | I-E (Pae) |
| AcrIE6 | P aeruginosa prophage | 79 | I-E (Pae) |
| AcrIE7 | P aeruginosa prophage | 106 | I-E (Pae) |
| AcrIF1 | P aeruginosa phage JBD30 | 78 | I-F (Pae, Pec) |
| AcrIF2 | P aeruginosa phage D3112 | 90 | I-F (Pae, Pec) |
| AcrIF3 | P aeruginosa phage JBD5 | 139 | I-F (Pae) |
| AcrIF4 | P aeruginosa phage JBD26 | 100 | I-F (Pae) |
| AcrIF5 | P aeruginosa phage JBD5 | 79 | I-F (Pae) |
| AcrIF6 | P aeruginosa prophage | 100 | I-E (Pae),/I-F (Pae, Pec) |
| AcrIF7 | P aeruginosa prophage | 67 | I-F (Pae, Pec) |
| AcrIF8 | Pectobacterium phage ZF40 | 92 | I-F (Pae, Pec) |
| AcrIF9 | Vibrio parahaemolyticus mobile | 68 | I-F (Pae, Pec) |
| | element | | |
| AcrIF10 | Shewanella xiamenensis | 97 | I-F (Pae, Pec) |
| | prophage | | |
| AcrIF11 | P aeruginosa prophage | 132 | I-F (Pae) |
| AcrIF12 | P aeruginosa mobile element | 124 | I-F (Pae) |
| AcrIF13 | Moraxella catarrhalis prophage | 115 | I-F (Pae) |
| AcrIF14 | Moraxella phage Mcat5 | 124 | I-F (Pae) |
| AcrIIA1 | Listeria monocytogenes | 149 | II-A (Lmo) |
| | prophage J0161a | | |
| AcrIIA2 | L monocytogenes prophage | 123 | II-A (Lmo, Spy) |
| | J0161a | | |
| AcrIIA3 | L monocytogenes prophage | 125 | II-A (Lmo) |
| | SLCC2482 | | |
| AcrIIA4 | L monocytogenes prophage | 87 | II-A (Lmo, Spy) |
| | J0161b | | |
| AcrIIA5 | Streptococcus thermophilus | 140 | II-A (Sth, Spy) |
| | phage D4276 | | |
| AcrIIA6 | S thermophilus phage D1811 | 183 | II-A (Sth) |
| AcrIIA7 | Metagenomic libraries from | 103 | II-A (Spy) |
| | human gut | | |
| AcrIIA8 | Metagenomic libraries from | 105 | II-A (Spy) |
| | human gut | | |
| AcrIIA9 | Metagenomic libraries from | 141 | II-A (Spy) |
| | human gut | | |
| AcrIIA10 | Metagenomic libraries from | 109 | II-A (Spy ) |
| | human gut | | |
| AcrIIC1 | Neisseria meningitidis | 85 | II-C (Nme, Cje, Geo, |
| | | | Hpa, Smu) |
| AcrIIC2 | N meningitidis prophage | 123 | II-C (Nme, Hpa, Smu) |
| AcrIIC3 | N meningitidis prophage | 116 | II-C (Nme, Hpa, Smu) |
| AcrIIC4 | Haemophilus parainfluenzae | 88 | II-C (Nme, Hpa, Smu) |
| | prophage | | |
| AcrIIC5 | Simonsiella muelleri prophage | 130 | II-C (Nme, Hpa, Smu) |
| AcrVA1 | M bovoculi prophage | 170 | V-A (Mb, As, Lb, Fn) |
| AcrVA2 | M bovoculi prophage | 322 | V-A (Mb) |
| AcrVA3 | M bovoculi prophage | 168 | V-A (Mb) |
| AcrVA4 | M bovoculi mobile element | 234 | V-A (Mb, Lb) |
| AcrVA5 | M bovoculi mobile element | 92 | V-A (Mb, Lb) |

Abbreviations in Table 1 are as follows: As, Acidaminococcus sp; Cje, Campylobacter jejuni; Fn, Francisella novicida; Geo, Geobacillus stearothermophilus; Hpa, Haemophilus parainfluenzae; Lb, Lachnospiraceae bacterium: Lno, Listeria monocytogenes; Mb, Moraxella bovoculi; Nme, 1Neisseria meningitidis; Pac, Pseudomonas aeruginosa; Pec, Pectobacterium atrosepticum; Sis, Sulfolobus islandicus; Spy, Streptococcus pyogenes; Sth, Streptococcus thermophilus.

Table 2 is a non-limiting list of known anti-CRISPR proteins and associated accession numbers.

TABLE 2

| ACR PROTEIN | ACCESSION NUMBER |
|---|---|
| Acr30-35 | Accession: 5UZ9_I |
| AcrE1 | Accession: 6AS4_C |
| AcrF1 | Accession: 6ANV_B |
| AcrF10 | Accession: 6ANW_C |
| AcrF2 | Accession: 6B47_K |
| AcrIIA1 | Accession: 5Y6A_B |
| AcrIIA2 | Accession: 6MCC_C |
| AcrIIA4 | Accession: 5VZL_C |
| AcrIIA5 | Accession: AVO22762.1 |
| AcrIIA6 | Accession: 6EYX_A |
| AcrIIA6 | Accession: AVO22749.1 |
| AcrIIA7-10 | As described in Uribe, RV., et |
| family | al. Cell Host and Microbe. |
| | 2019. |
| AcrIIC1 | Accession: 5VGB_B |
| AcrIIC2 | Accession: 6J9M_D |
| AcrIIC3 | Accession: 6J9N_B |
| AcrIIC4 | As described in Lee, J., et al. |
| AcrIIC5 | 2018. mBio. |
| AcrIID1 | Accession: 6EXP_F |
| AcrVA1 | As described in Dong, L., et |
| AcrVA4 | al. 2019. Nature Struct and |
| AcrVA5 | Mol Biol. |

One exemplary anti-CRISPR protein, AcrIIC1, is an 85 amino acid protein that inactivates a wide range of type IIC Cas9 orthologs by binding to and conformationally restraining the conserved HNH domain of Cas. AcrIIC1 is a broad-spectrum Cas9 inhibitor that prevents DNA cutting by multiple divergent Cas9 orthologs through direct binding to the conserved HNH catalytic domain of Cas9. The amino acid sequence of SEQ ID NO: 6 encodes AcrIIC1.

(SEQ ID NO: 6)
MAKEVFKLKPELVTYKGCGWALACIKDGEIIDLTYVRDLGIEEYDENFD
GLEPEIIYYDVVASQACKEVAYRYEEMGEFTFGLCSCWEFNVM

Another anti-CRISPR protein useful in the invention described herein is AcrIIA4, a broad-spectrum Cas9 inhibitor, including the widely used Cas9 ortholog from Streptococcus pyogenes (SpyCas9). AcrIIA4 interferes with DNA recognition but has no effect on preformed Cas9-sgRNA-DNA complexes. The amino acid sequence of SEQ ID NO: 7 encodes AcrIIA4.

(SEQ ID NO: 7)
MNINDLIREIKNKDYTVKLSGTDSNSITQLIIRVNNDGNEYVISESENE
SIVEKFISAFKNGWNQEYEDEEEFYNDMQTITLKSELN.

Another exemplary anti-CRISPR protein is AcrIIA2, which interacts with the protospacer adjacent motif (PAM) recognition residues of SpyCas9, preventing target double-stranded DNA (dsDNA) detection. The amino acid sequence of SEQ ID NO: 8 encodes AcrIIA2.

(SEQ ID NO: 8)
MTLTRAQKKYAEAMHEFINMVDDFEESTPDFAKEVLHDSDYVVITKNEK
YAVALCSLSTDECEYDTNLYLDEKLVDYSTVDVNGVTYYINIVETNDID
DLEIATDEDEMKSGNQEIILKSELK

In one embodiment, the anti-CRISPR agent (e.g., an anti-CRISPR protein, peptide, or intrabody) can be delivered via plasmid/DNA/RNA/Protein transfection methods (e.g., transfection, liposome delivery, electroporation, etc.) as single element or part of another viral vector production system (e.g. cloned into the helper plasmid used in virus production), or be constitutively expressed in a stable cell line useful for viral vector production. It can be used as a single protein or in combination with other anti-gene-editing proteins, e.g., multi-anti-CRISPR-broad-spectrum-partners, to target multiple Cas domains and prevent DNA binding, gRNA/Cas9 binding, etc.

The HNH domain of a Cas protein is a nuclease domain that functions to cleave the DNA strand complementary to the guide RNA (gRNA) via a one-metal-ion dependent mechanism. The HNH nuclease domain (residues 775-908 of cas9) lies in between the RuvC II-III motifs and forms only a few contacts with the rest of the protein. HNH nuclease domain comprises a two-stranded antiparallel β-sheet (β12 and β13) flanked by four α-helices (α35-α38). The HNH nuclease has a ββα-metal fold that comprises the active site, and there are many structural similarities between this nuclease and others that contain the ββα-metal fold, such as phage T4 endonuclease VII (Endo VII) and Vibrio vulnificus nuclease.

Cas9 proteins vary widely in both sequence and size but all known Cas9 enzymes contain an HNH domain that cleaves the DNA strand complementary to the guide RNA sequence (target strand), and a RuvC nuclease domain required for cleaving the noncomplementary strand (non-target strand), yielding double strand DNA breaks (DSBs). The Cas9-type HNH nuclease domain contains a two-stranded antiparallel β sheet flanked by two α-helices on each side.

Correct conformational formation is essential for proper function of the HNH domain; point mutations in the active site result in an altered conformation, inactivating the HNH domain and inhibiting the nuclease function of Cas. The HNH domain of Cas is further described in, for example, Nishimasu, et al., 2014; Jinnek, et al., 2014; Biertumpfel, et al., 2007; and Li et al., 2003; the contents of which are incorporated herein by reference in their entireties.

It is specifically contemplated herein that anti-CRISPR peptides identified via in-silico screening or experimental methods (including but not limited to screening phage-displayed random peptide libraries) can be used to inactivate the CRISPR-Cas gene-editing system. A peptide useful in this invention will bind to and inhibit the nuclease activity of the gene-editing protein. For example, a peptide can bind to the HNH domain of a Cas protein and induce an inhibitory conformational change. Further, functional fragments of anti-CRISPR proteins can be used to inhibit the nuclease activity of a gene-editing protein. Functional fragments comprise at least 85%, 96%, 87%, 88%, 89%, 90%, 91%, 92%, 93%, 94%, 95%, 96%, 97%, 98%, 99%, or more sequence identity to the full length sequence of an anti-CRISPR protein, and comprise at least 50% of the same function as the full length protein (e.g., inhibiting the nuclease activity of a gene-editing protein).

In various embodiments, the inhibitor of a gene-editing protein is an antibody or antigen-binding fragment thereof, or an antibody reagent that is specific for HNH domain of a Cas protein. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like. Monoclonal antibodies are prepared using methods well known to those of skill in the art.

In one embodiment, the antibody used is a single chain antibody (scAb), a Fab′ fragment, or a single domain antibody (dAB). Artisan can readily screen for, for example, a scAb by screening a phage display antibody library, e.g., mouse or human, for a single chain variable fragment that binds to the desired domain of a gene-editing protein to interfere with such protein, for example the HNH domain of Cas.

In certain embodiments, the inhibitor of the gene-editing protein is an intrabody, i.e. an intracellular antibody that functionally binds a target within a cell (See, generally, Hood et al., Immunology, Benjamin, N.Y., 2ND ed. (1984), Harlow and Lane, Antibodies. A Laboratory Manual, Cold Spring Harbor Laboratory (1988); Hunkapiller and Hood, Nature, 323, 15-16 (1986); and Rondon and Marasco, Annu Rev Microbiol, 51:257-83 (1997); U.S. Pat. Nos. 6,004,940; and 5,581,829; which are incorporated herein by reference in their entireties). Methods for intrabody production are well known to those of skill in the art, e.g. as described in WO 2002/086096, which is incorporated herein by reference in its entirety. Antibodies will usually bind with at least a KD of about 1 mM, more usually at least about 300 μM, typically at least about 10 μM, more typically at least about 30 μM, preferably at least about 10 μM, and more preferably at least about 3 μM or better.

Intrabodies that inhibit a gene-editing protein, for example, Cas9, can be identified via high-throughput screening platforms that e.g., measure a fluorescence-polarization-based primary screening assay for probing and identifying SpCas9-PAM binding, and secondary measurements to assess SpCas9 activity using gain or loss of fluorescent signal. For example, if an intrabody inhibits SpCas9 binding to PAM, thus inhibiting the SpCas9's capability to cleave DNA, one would expect a loss of signal in this high-throughput screen. Such screening platform can be further reviewed in, e.g., Maji, B., et al. Cell. 2019; Fan, Shelly. “The hunt for a CRISPR Antidote Just Heated Up.” 15 May 2019. SingularityHub. Web. Accessed 2 Jul. 2019; and Cell Press. “Drugs that block CRISPR-Cas9 genome editing identified.” ScienceDaily. 2 May 2019. Accessed 2 Jul. 2019; the contents of which are incorporated herein in its entirety.

In one embodiment, antibody binding of the HNH domain results in a conformational change to the structure of the Cas protein, e.g., in the HNH domain. Conformational changes in a protein structure can be determined, e.g., by generating a crystal structure of a given protein with and without the bound antibody. In one embodiment, antibody binding of the HNH domain inactivates the activity, e.g., the nuclease activity, of the Cas protein. For example, at least 5%, 10%, 20%, 30%, 40%, 50%, 60%, 70%, 80%, 90%, 99% or more of the activity, e.g., the nuclease activity, is inactivated following antibody binding to the HNH domain. A number of assays that can be used to assess the nuclease activity of a Cas protein are known in the art. As one example, the amount of nuclease activity can be determined by employing whole genome sequencing in a cell expressing a Cas protein and an antibody that binds the HNH domain of Cas, and a cell expressing only a Cas protein. One would expect to find a higher incidence of mutations (e.g., on-target and off-target) in a cell having nuclease activity. Further, assays for broadly detecting nuclease activity are known in the art. For example, a CRISPRuTest™ Functional Cas9 Activity Assay can be used to measure Cas9 nuclease activity in any mammalian cell system. The CRISPRuTest Kit provides a FACS-based assay to assess the Cas9 nuclease activity in cells expressing Streptococcus pyogenes Cas9. This kit contains pseudoviral packaged lentiviral constructs with a green fluorescent protein (GFP) gene whose fluorescence is disrupted in cells with active Cas9 nuclease activity. Additionally, cell-free assays have been developed to measure Cas nuclease activity. Cell-free assays for measuring Cas nuclease activity is further described in, for example, Cox, K. J., et al. Chem Sci, 2019, which is incorporated herein in its entirety. It is specifically contemplated that any method known in the art that measures nuclease activity of a gene-editing protein can be used herein to assess the antibody's ability to inhibit nuclease activity of a gene-editing protein.

One aspect described herein provides an antibody that binds to the HNH domain of a Cas protein. In various embodiments, binding of the HNH domain inhibits the nuclease activity of a Cas protein.

An exemplary commercially available antibody that binds the HNH domain of a Cas protein include, but is not limited to, Anti-Cas9 Antibody, D10A/H840A Mutant (Millipore Sigma; Burlington, Mass.).

One aspect described herein provides an antibody generated by an epitope comprising an HNH domain. In one embodiment, the antibody is generated by a peptide containing only one epitope, wherein the only one epitope is in the HNH domain. One skilled of the art will be able to generate an antibody by an epitope using standard techniques, e.g., as described herein.

### Gene-Editing Proteins

In one embodiment, the gene-editing protein is a nuclease, e.g., a CRISPR-associated nuclease, meganuclease, zinc finger nuclease, transcription activator-like effector nuclease, endonuclease, or an exonuclease.

As used herein, the term “nuclease” refers to molecules which possesses activity for DNA cleavage. Particular examples of nuclease agents for use in the methods disclosed herein include RNA-guided CRISPR-Cas9 system, zinc finger proteins, meganucleases, TAL domains, TALENs, yeast assembly, recombinases, leucine zippers, CRISPR/Cas, endonucleases, and other nucleases known to those in the art. Nucleases can be selected or designed for specificity in cleaving at a given target site. For example, nucleases can be selected for cleavage at a target site that creates overlapping ends between the cleaved polynucleotide and a different polynucleotide. Nucleases having both protein and RNA elements, such as in CRISPR-Cas9, can be supplied with the agents already complexed as a nuclease, or can be supplied with the protein and RNA elements separate, in which case they complex to form a nuclease in the reaction mixtures described herein. In one embodiment, the gene-editing protein is the Cas9 nuclease. In another embodiment, a nuclease other than Cas9 is used.

As used herein, the term “recognition site for a nuclease” refers to a DNA sequence at which a nick or double-strand break is induced by a nuclease. The recognition site for a nuclease can be endogenous (or native) to the cell or the recognition site can be exogenous to the cell. In specific embodiments, the recognition site is exogenous to the cell and thereby is not naturally occurring in the genome of the cell. In still further embodiments, the recognition site is exogenous to the cell and to the polynucleotides of interest that one desires to be positioned at the target locus. In further embodiments, the exogenous or endogenous recognition site is present only once in the genome of the host cell. In specific embodiments, an endogenous or native site that occurs only once within the genome is identified. Such a site can then be used to design nuclease agents that will produce a nick or double-strand break at the endogenous recognition site.

The length of the recognition site can vary, and includes, for example, recognition sites that are about 30-36 bp for a zinc finger nuclease (ZFN) pair (i.e., about 1518 bp for each ZFN), about 36 bp for a Transcription Activator-Like Effector Nuclease (TALEN), or about 20 bp for a CRISPR/Cas9 guide RNA.

The gene-editing protein can be any nuclease that induces a nick or double-strand break into a desired recognition site can be used in the methods disclosed herein. A naturally-occurring or native nuclease can be employed so long as the nuclease agent induces a nick or double-strand break in a desired recognition site. Alternatively, a modified or engineered nuclease agent can be employed. An “engineered nuclease” comprises a nuclease that is engineered (modified or derived) from its native form to specifically recognize and induce a nick or double-strand break in the desired recognition site. Thus, an engineered nuclease agent can be derived from a native, naturally-occurring nuclease agent or it can be artificially created or synthesized. The modification of the nuclease agent can be as little as one amino acid in a protein cleavage agent or one nucleotide in a nucleic acid cleavage agent. In some embodiments, the engineered nuclease induces a nick or double-stranded break in a recognition site, wherein the recognition site was not a sequence that would have been recognized by a native (non-engineered or non-modified) nuclease agent. Producing a nick or double-strand break in a recognition site or other DNA can be referred to herein as “cutting” or “cleaving” the recognition site or other DNA.

These breaks can then be repaired by the cell in one of two ways: non-homologous end joining and homology-directed repair (homologous recombination). In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence can be used as a template for repair of the cleaved target DNA sequence, resulting in the transfer of genetic information from the donor polynucleotide to the target DNA. Therefore, new nucleic acid material may be inserted/copied into the site. The modifications of the target DNA due to NHEJ and/or homology-directed repair can be used for gene correction, gene replacement, gene tagging, transgene insertion, nucleotide deletion, gene disruption, gene mutation, etc.

In one embodiment, the gene-editing protein is a CRISPR-associated nuclease. The native prokaryotic CRISPR-associated nuclease system comprises an array of short repeats with intervening variable sequences of constant length (i.e., clusters of regularly interspaced short palindromic repeats), and CRISPR-associated (“Cas”) nuclease proteins. The RNA of the transcribed CRISPR array is processed by a subset of the Cas proteins into small guide RNAs, which generally have two components as discussed below. There are at least three different systems: Type I, Type II and Type III. The enzymes involved in the processing of the RNA into mature crRNA are different in the 3 systems. In the native prokaryotic system, the guide RNA (“gRNA”) comprises two short, non-coding RNA species referred to as CRISPR RNA (“crRNA”) and trans-acting RNA (“tracrRNA”). In an exemplary system, the gRNA forms a complex with a nuclease, for example, a Cas nuclease. The gRNA: nuclease complex binds a target polynucleotide sequence having a protospacer adjacent motif (“PAM”) and a protospacer, which is a sequence complementary to a portion of the gRNA. The recognition and binding of the target polynucleotide by the gRNA: nuclease complex induces cleavage of the target polynucleotide. The native CRISPR-associated nuclease system functions as an immune system in prokaryotes, where gRNA: nuclease complexes recognize and silence exogenous genetic elements in a manner analogous to RNAi in eukaryotic organisms, thereby conferring resistance to exogenous genetic elements such as plasmids and phages. It has been demonstrated that a single-guide RNA (“sgRNA”) can replace the complex formed between the naturally-existing crRNA and tracrRNA.

Any CRISPR-associated nuclease can be used in the system and methods of the invention. CRISPR nuclease systems are known to those of skill in the art, e.g. see Patents/applications U.S. Pat. No. 8,993,233, US 2015/0291965, US 2016/0175462, US 2015/0020223, US 2014/0179770, U.S. Pat. Nos. 8,697,359; 8,771,945; 8,795,965; WO 2015/191693; U.S. Pat. No. 8,889,418; WO 2015/089351; WO 2015/089486; WO 2016/028682; WO 2016/049258; WO 2016/094867; WO 2016/094872; WO 2016/094874; WO 2016/112242; US 2016/0153004; US 2015/0056705; US 2016/0090607; US 2016/0029604; 8,865,406; 8,871,445; each of which are incorporated by reference in their entirety.

In one embodiment, the gene-editing protein is a meganuclease. Meganucleases have been classified into four families based on conserved sequence motifs, the families are the LAGLIDADG (SEQ ID NO: 1), GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. Meganuclease domains, structure and function are known, see for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764. In some examples a naturally occurring variant, and/or engineered derivative meganuclease is used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames etal., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346, which are incorporated herein by reference in their entireties.

Any meganuclease can be used herein, including, but not limited to, I-Scel, I-Scell, 1-SceIII, I-SceIV, I-SceV, I-SceVI, I-SceVII, I-Ceul, I-CeuAIIP, I-Crel, 1-CrepsbIP, I-CrepsbllP, 1-CrepsbIIIP, 1-CrepsbIVP, I-Tlil, I-Ppol, PI-PspI, F-Scel, F-Scell, F-Suvl, F-TevI, F-TevII, I-Amal, I-Anil, I-Chul, I-Cmoel, I-Cpal, I-CpaII, I-CsmI, I-Cvul, I-CvuAIP, I-Ddil, I-DdiII, I-Dirl, I-Dmol, I-Hmul, I-HmuII, I—HsNIP, I-Llal, I-Msol, I-Naal, I-NanI, I-NcIIP, I-NgrIP, I-Nitl, I-Njal, I-Nsp236IP, I-PakI, I-PboIP, I-PcuIP, I-PcuAI, I-PcuVI, I-PgrlP, I-PobIP, I-Porl, I-PorIIP, I-PbpIP, I-SpBetaIP, I-Scal, I-SexIP, I-SneIP, I-Spoml, I-SpomCP, I-SpomIP, I-SpomlIP, I-SquIP, I-Ssp68O3I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MtuI, PI-MtuHIP PI-MtuHIIP, PI-PfuI, PI-PfuII, PI-PkoI, PI-PkoII, PI-Rma43812IP, PI-SpBetaIP, PI-SceI, PI-Tful, PI-TfuII, PI-Thyl, PI-Tlil, PI-TliII, or any active variants or fragments thereof.

In one embodiment, the meganuclease recognizes double-stranded DNA sequences of 12 to 40 base pairs. In one embodiment, the meganuclease recognizes one perfectly matched target sequence in the genome. In one embodiment, the meganuclease is a homing nuclease. In one embodiment, the homing nuclease is a LAGLIDADG (SEQ ID NO: 1) family of homing nuclease. In one embodiment, the LAGLIDADG (SEQ ID NO: 1) family of homing nuclease is selected from I-Scel, I-Crel, and I-Dmol.

In one embodiment, the gene-editing protein is a zinc-finger nuclease (ZFN). In one embodiment, each monomer of the ZFN comprises 3 or more zinc finger-based DNA binding domains, wherein each zinc finger-based DNA binding domain binds to a 3 bp subsite. In other embodiments, the ZFN is a chimeric protein comprising a zinc finger-based DNA binding domain operably linked to an independent nuclease. In one embodiment, the independent endonuclease is a FokI endonuclease. In one embodiment, the nuclease agent comprises a first ZFN and a second ZFN, wherein each of the first ZFN and the second ZFN is operably linked to a FokI nuclease subunit, wherein the first and the second ZFN recognize two contiguous target DNA sequences in each strand of the target DNA sequence separated by about 5-7 bp spacer, and wherein the FokI nuclease subunits dimerize to create an active nuclease that makes a double strand break. See, for example, US20060246567; US20080182332; US20020081614; US20030021776; WO/2002/057308A2; US20130123484; US20100291048; WO/2011/017293A2; and Gaj etal. (2013) Trends in Biotechnology, 31(7):397-405, each of which is herein incorporated by reference in their entireties.

In one embodiment, the gene-editing protein is a Transcription Activator-Like Effector Nuclease (TALEN). TALENs are a class of sequence-specific nucleases that can be used to make double-stranded breaks at specific target sequences in the genome of a prokaryotic or eukaryotic organism. TALENs are created by fusing a native or engineered transcription activator-like (TAL) effector, or functional part thereof, to the catalytic domain of an endonuclease, such as, for example, Fokl. The unique, modular TAL effector DNA binding domain allows for the design of proteins with potentially any given DNA recognition specificity. Thus, the DNA binding domains of the TALENs can be engineered to recognize specific DNA target sites and thus, used to make double-stranded breaks at desired target sequences. See, WO 2010/079430; Morbitzer et al. (2010) PNAS 10.1073/pnas.1013133107; Scholze &amp; Boch (2010) Virulence 1:428-43; Christian et al. Genetics (2010) 186:757-761; Li et al. (2010) Nuc. Acids Res. (2010) doi:10.1093/nar/gkg704; and Miller et al. (2011) Nature Biotechnology 29:143-148; all of which are herein incorporated by reference in their entireties.

Examples of suitable TALENs, and methods for preparing suitable TALENs, are disclosed, e.g., in US Patent Application No. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1 (each hereby incorporated by reference in their entireties). In various embodiments, TALENs are engineered to cut in or near a target nucleic acid sequence in, e.g., a genomic locus of interest, wherein the target nucleic acid sequence is at or near a sequence to be modified by a targeting vector. The TALENs suitable for use with the various methods and compositions provided herein include those that are specifically designed to bind at or near target nucleic acid sequences to be modified by targeting vectors as described herein.

In one embodiment, each monomer of the TALEN comprises 33-35 TAL repeats that recognize a single base pair via two hypervariable residues. In one embodiment, the nuclease agent is a chimeric protein comprising a TAL repeat-based DNA binding domain operably linked to an independent nuclease. In one embodiment, the independent nuclease is a FokI endonuclease. In one embodiment, the nuclease agent comprises a first TAL-repeat-based DNA binding domain and a second TAL-repeat-based DNA binding domain, wherein each of the first and the second TAL-repeat-based DNA binding domain is operably linked to a FokI nuclease subunit, wherein the first and the second TAL-repeat-based DNA binding domain recognize two contiguous target DNA sequences in each strand of the target DNA sequence separated by a spacer sequence of varying length (12-20 bp), and wherein the FokI nuclease subunits dimerize to create an active nuclease that makes a double strand break at a target sequence.

In one embodiment, the gene-editing protein is a restriction endonuclease (i.e., restriction enzymes), which include Type I, Type II, Type III, and Type IV endonucleases. Type I and Type III restriction endonucleases recognize specific recognition sites, but typically cleave at a variable position from the nuclease binding site, which can be hundreds of base pairs away from the cleavage site (recognition site). In Type II systems the restriction activity is independent of any methylase activity, and cleavage typically occurs at specific sites within or near to the binding site. Most Type II enzymes cut palindromic sequences, however Type IIa enzymes recognize non-palindromic recognition sites and cleave outside of the recognition site, Type lib enzymes cut sequences twice with both sites outside of the recognition site, and Type IIs enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res 31:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.).

In one embodiment, the gene-editing protein is an exonuclease. Exonucleases are enzyme that function by cleaving nucleotides are the end of a polynucleotide chain via a hydrolyzing reaction that breaks phosphodiester binds at either the 5′ or 3′ ends. An exonuclease can be endogenous or exogenous to the cell. Nonlimiting examples of native exonucleases include exonuclease I, exonuclease II, exonuclease III, exonuclease IV, exonuclease V, and exonuclease VIII.

In another embodiment, the gene-editing protein is Natronobacterium gregoryi Argonaute protein (NgAgo). NgAgo is an endonuclease that utilizes a pair of 5′ phosphorylated, reverse complementary guide DNAs or RNAs (e.g., siRNA) to target and cut a target nucleic acid (e.g., genomic DNA). Importantly, Argonaute proteins do not a requite a motif (e.g., PAM) in the sequence of the target nucleic acid.

Sequences for NgAgo are known in the art. For example, NgAgo can have the sequence of SEQ ID NO: 2.

SEQ ID NO: 2 is an amino acid sequence encoding NgAgo (NCBI accession number: ANC90309.1).

| (SEQ ID NO: 2) | |
|---|---|
| 1 | mtvidldstt tadeltsght ydisvtltgv ydntdeqhpr mslafeqdng erryitlwkn |
| 61 | ttpkdvftyd yatgstyift nidyevkdgy enltatyqtt venataqevg ttdedetfag |
| 121 | gepldhhldd alnetpddae tesdsghvmt sfasrdqlpe wtlhtytlta tdgaktdtey |
| 181 | arrtlaytvr qelytdhdaa pvatdglmll tpeplgetpl dldcgvrvea detrtldytt |
| 241 | akdrllarel veeglkrslw ddylvrgide vlskepvltc defdlheryd lsvevghsgr |
| 301 | aylhinfrhr fvpkltladi dddniypglr vkttyrprrg hivwglrdec atdslntlgn |
| 361 | qsvvayhrnn qtpintdlld aieaadrrvv etrrqghgdd avsfpqella vepnthqikq |
| 421 | fasdgfhqqa rsktrlsasr csekaqafae rldpvrlngs tvefssefft gnneqqlrll |
| 481 | yengesvltf rdgargahpd etfskgivnp pesfevavvl peqqadtcka qwdtmadlln |
| 541 | qagapptrse tvqydafssp esislnvaga idpsevdaaf vvlppdqegf adlasptety |
| 601 | delkkalanm giysqmayfd rfrdakifyt rnvalgllaa aggvaftteh ampgdadmfi |
| 661 | gidvsrsype dgasgqinia atatavykdg tilghsstrp qlgeklqstd vrdimknail |
| 721 | gyqqvtgesp thivihrdgf mnedldpate flneqgveyd iveirkqpqt rllavsdvqy |
| 781 | dtpvksiaai nqnepratva tfgapeylat rdggglprpi qiervagetd ietltrqvyl |
| 841 | lsqshiqvhn starlpitta yadqasthat kgylvqtgaf esnvgfl |

The expression and proper folding of NgAgo can be sensitive to conditions such as salt concentration. NgAgo can be expressed in a cell with a high concentration of salt. NgAgo can be expressed in a cell with a low or moderate salt concentration and the resultant expressed NgAgo protein can be divided into soluble and insoluble fractions. Functional NgAgo can be found in the soluble fraction.

Guide DNA sequences for a target nucleic acid can any 20-30 base pair (bp) sequence in the target nucleic acid. For example, 22 bp, 24 bp, 26 bp, 28 bp, or 30 bp.

In one embodiment, the gene-editing protein is Artificial restriction DNA cutter (ARCUT). Non-restriction enzyme methodology termed artificial restriction DNA cutter (ARCUT) can be used to edit chromosomal DNA of the cell is using the materials and methods described herein. This method uses pseudo-complementary peptide nucleic acid (pcPNA) to specify the cleavage site within the chromosome or the telomeric region. Once pcPNA specifies the site, excision here is carried out by cerium (CE) and EDTA (chemical mixture), which performs the splicing function. Furthermore, the technology uses a DNA ligase that can later attach any desirable DNA within the spliced site (see e.g. Komiyama M. Chemical modifications of artificial restriction DNA cutter (ARCUT) to promote its in vivo and in vitro applications. Artif DNA PNA XNA. 2014; 5:e1112457).

Various promoters that direct expression of the inhibitor of a gene-editing protein can be used in the gene-editing system described herein. Examples include, but are not limited to, constitutive promoters, repressible promoters, and/or inducible promoters, some non-limiting examples of which include viral promoters (e.g., CMV, SV40), tissue specific promoters (e.g., muscle MCK), heart (e.g., NSE), eye (e.g., MSK) and synthetic promoters (SP1 elements) and the. chicken beta actin promoter (CB or CBA). The promoter can be present in any position on where it is in operable association with the nuclease sequence.

Inducible Promoters

An inducible promoter may be a promoter induced by the presence of an inducer, the absence of a repressor, or any other suitable physical or chemical condition that induces transcription from the inducible promoter. The terms “inducer”, “inducing conditions” and suchlike should be understood accordingly. The use of an inducible promoter further reduces the possibility of undesired expression of the Cas proteins.

By way of non-limiting example, an inducible promoter for use in embodiments of the invention may be a small molecule-inducible promoter, a tetracycline-regulatable (e.g. inducible or repressible) promoter, an alcohol-inducible promoter, a steroid-inducible promoter, a mifepristone (RU486)-inducible promoter, an ecdysone-inducible promoter, a rapamycin-inducible promoter, a metallothionein-inducible promoter, a hormone-inducible promoter, a cumate-inducible promoter, a temperature-inducible promoter, a pH-inducible promoter and a metal-inducible promoter.

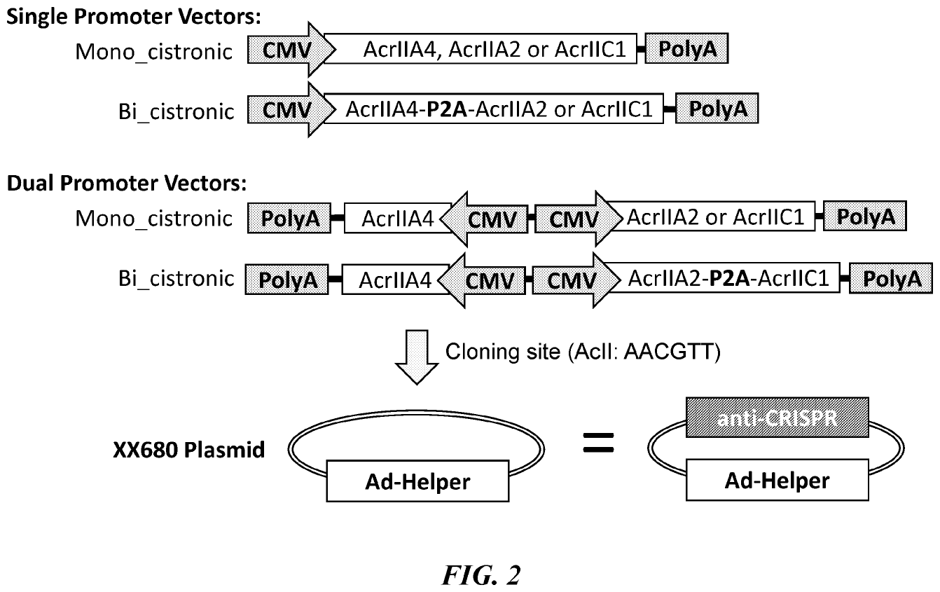

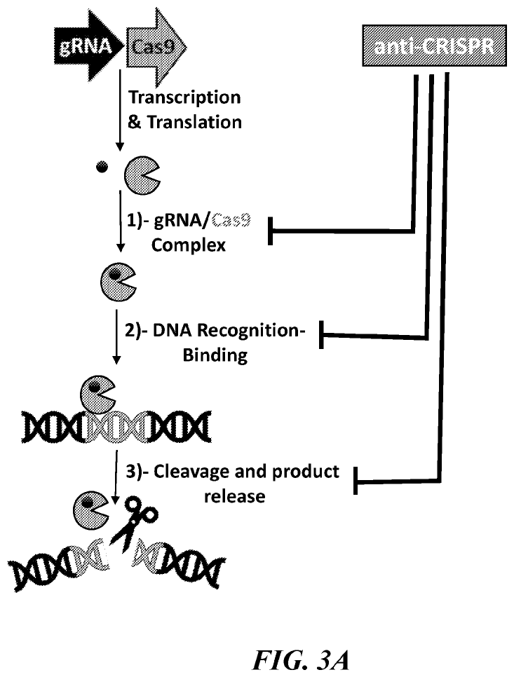

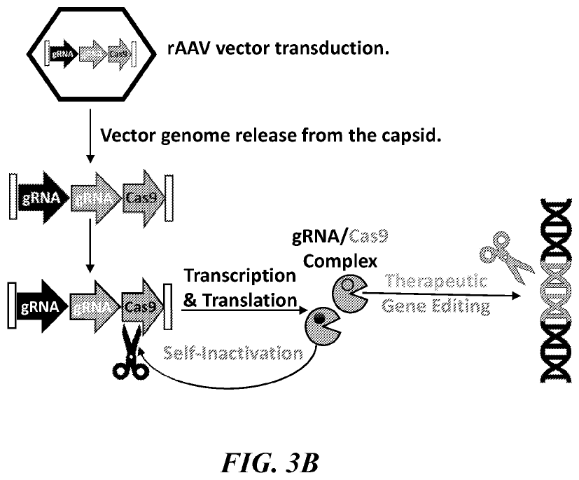

Temperature-Inducible Promoters—The inducible promoter may be induced by reduction of temperature, e.g. a cold-shock responsive promoter. In some embodiments, the inducible promoter is a synthetic cold-shock responsive promoter derived from the S1006a gene (calcyclin) of CHO cells. The temperature sensitivity of the S1006a gene (calcyclin) promoter was identified by Thaisuchat et al., 2011 (Thaisuchat, H. et al. (2011) ‘Identification of a novel temperature sensitive promoter in cho cells’, BMC Biotechnology, 11. doi: 10.1186/1472-6750-11-51), which is incorporated herein by reference. In some embodiments, the inducible promoter is one of the synthetic cold-shock responsive promoters shown in FIG. 2 of Thaisuchat et al., 2011. These promoters are induced by decrease of temperature as shown in FIG. 3 of Thaisuchat et al., 2011. Most of these synthetic promoter constructs show expression similar to the known promoter SV40 at 37° C. and are induced by 2-3 times when the temperature is reduced to 33° C. In some embodiments, the inducible promoter is sps5 from FIG. 2 of Thaisuchat et al., 2011. In some preferred embodiments, the inducible promoter is sps8 from FIG. 2 of Thaisuchat et al., 2011.

pH-Inducible Promoters—The inducible promoter may be induced by reduction or increase of pH to which cells comprising the promoter are exposed. Suitably, the inducible promoter may be induced by reduction of pH, i.e. a promoter inducible under acidic conditions. Suitable acid-inducible promoters are described in Hou et al., 2016 (Hou, J. et al. (2016) ‘Isolation and functional validation of salinity and osmotic stress inducible promoter from the maize type-II H+-pyrophosphatase gene by deletion analysis in transgenic tobacco plants’, PLoS ONE, 11(4), pp. 1-23. doi: 10.1371/journal.pone.0154041), which is incorporated herein by reference.

In some embodiments, the inducible promoter is a synthetic promoter inducible under acidic conditions derived from the YGP1 gene or the CCW14 gene. The inducibility by acidic conditions of the YGP1 gene or the CCW14 gene was studied and improved by modifying transcription factor binding sites by Rajkumar et al., 2016 (Rajkumar, A. S. et al. (2016) ‘Engineering of synthetic, stress-responsive yeast promoters’, 44(17). doi: 10.1093/nar/gkw553), which is incorporated herein by reference. In some embodiments, the inducible promoter is one of the synthetic promoter inducible under acidic conditions in FIG. 1A, 2A, 3A and 4A of Rajkumar et al., 2016. These promoters are induced by decrease of pH as shown in FIG. 1B, 2B, 3B and 4B of Rajkumar et al., 2016. Most of these synthetic promoters are induced by up to 10-15 times when the reduced from pH 6 to pH 3. In some preferred embodiments, the inducible promoter is YGP1pr from FIG. 1 of Rajkumar et al., 2016. In other preferred embodiments, the inducible promoter is YGP1pr from FIG. 1 of Rajkumar et al., 2016.

Osmolarity-Inducible Promoters—The inducible promoter may be osmolarity-induced. Suitable promoters induced by osmolarity are described in Zhang et al. (Molecular Biology Reports volume 39, pages 7347-7353(2012)) which is incorporated herein by reference.

Carbon Source-Inducible Promoters—The inducible promoter may be induced by addition of a specific carbon source, e.g. a non-sugar carbon source. Alternatively, the inducible promoter may be induced by withdrawal or the absence of a carbon source. Suitable promoters induced by the presence or absence of various carbon sources are described in Weinhandl et al., 2014 (Weinhandl, K. et al. (2014) ‘Carbon source dependent promoters in yeasts’, Microbial Cell Factories, 13(1), pp. 1-17. doi: 10.1186/1475-2859-13-5), which is incorporated herein by reference.

Alcohol (e.g. Ethanol)-Inducible Promoters—The inducible promoter may be induced by addition of ethanol. Suitable promoters induced by ethanol are described in Matsuzawa et al. (Applied Microbiology and Biotechnology volume 97, pages 6835-6843(2013)), which is incorporated herein by reference.

Amino Acid-Inducible Promoters—The inducible promoters may be induced by addition of one or more amino acids. Suitably, the amino acid may be an aromatic amino acid. Suitably, the amino acid may be GABA (gamma aminobutyric acid), which is also a neurotransmitter. Suitable promoter induced by aromatic amino acids and GABA are described in Kim et al. (Applied Microbiology and Biotechnology, volume 99, pages 2705-2714(2015)) which is incorporated herein by reference.

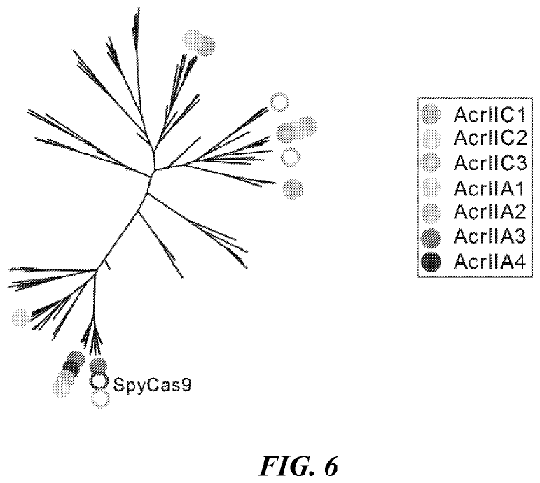

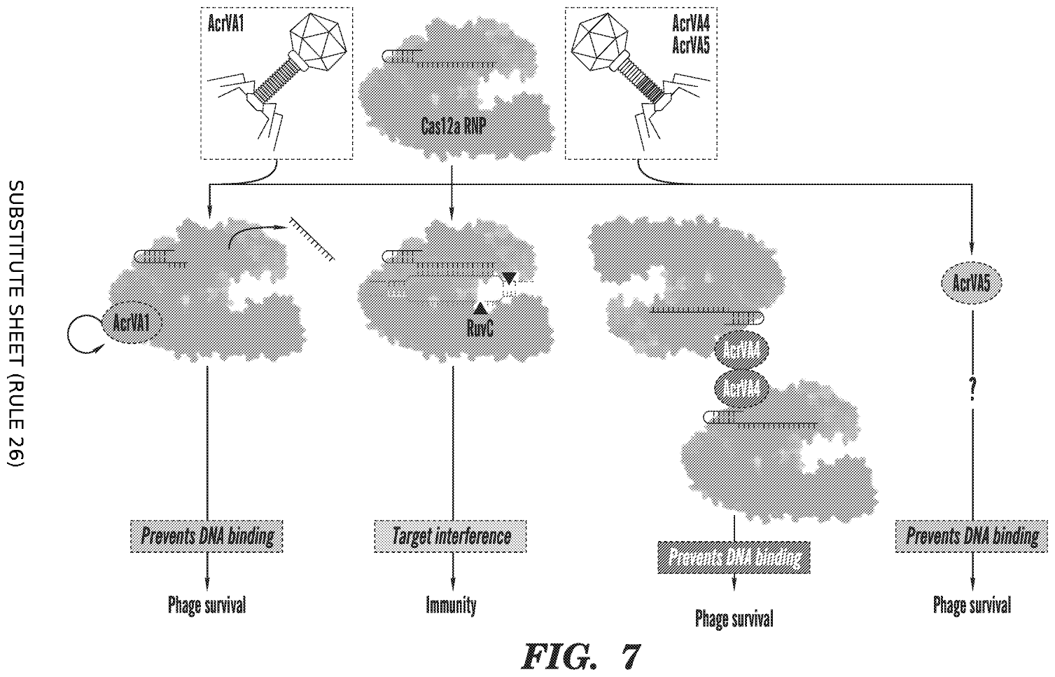

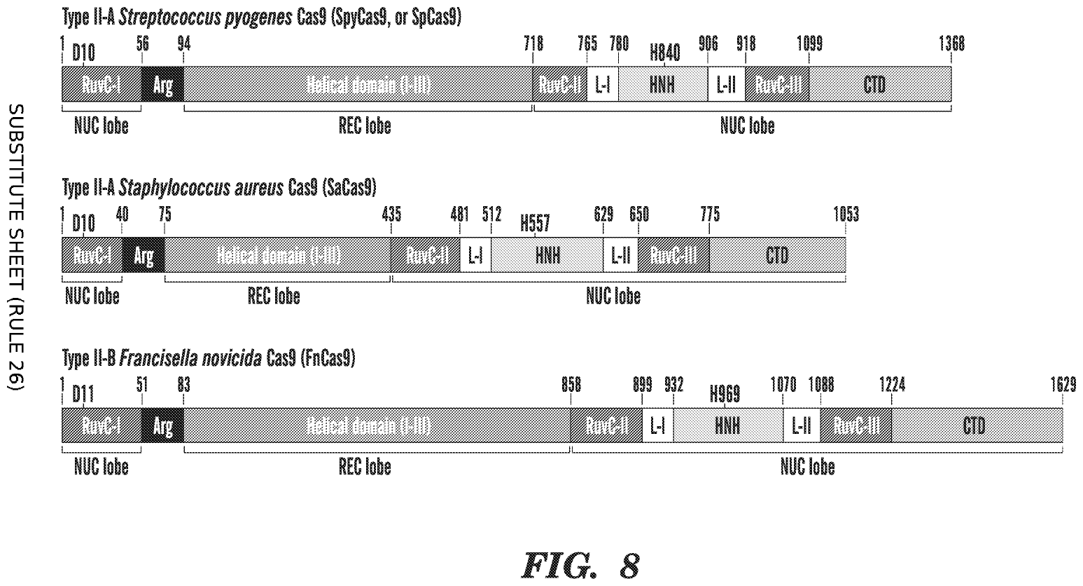

Hormone (e.g. Ecdysone)-Inducible Promoters—The inducible promoter may be the induced by a steroid hormone. Suitably, the steroid hormone may be ecdysone. A mammalian ecdysone-inducible system was created by No, Yao and Evans (No, D., Yao, T. P. and Evans, R. M. (1996) ‘Ecdysone-inducible gene expression in mammalian cells and transgenic mice’, Proceedings of the National Academy of Sciences of the United States of America, 93(8), pp. 3346-3351. doi: 10.1073/pnas.93.8.3346), which is incorporated herein by reference. Expression of a modified ecdysone receptor in mammalian cells allows expression from an ecdysone responsive promoter to be induced upon addition of ecdysone as shown in FIG. 2 of No, Yao and Evans, 1996. This system showed lower basal activity and higher inducibility than the tetracycline-inducible system as shown in FIG. 6 of No, Yao and Evans, 1996. A suitable commercially available inducible system is available from Agilent technologies and is described in Agilent Technologies (2015) ‘Complete Control Inducible Mammalian Expression System Instruction Manual’, 217460, which is incorporated herein by reference.

Tetracycline-Regulated Promoters—In some embodiments, the promoter may be induced by the presence or absence of tetracycline or its derivatives.

A suitable promoter induced in the absence of tetracycline or its derivatives is the promoter in the tet-OFF system. In the tet-OFF system, tetracycline-controlled transactivator (tTA) allows transcriptional activation of a tTA-dependent promoter in the absence of tetracycline or its derivatives. tTA and the tTA-dependent promoter were initially created by Gossen and Bujard, 1992 (Gossen, M. and Bujard, H. (1992) ‘Tight control of gene expression in mammalian cells by tetracycline-responsive promoters’, Proceedings of the National Academy of Sciences of the United States of America, 89(12), pp. 5547-5551. doi: 10.1073/pnas.89.12.5547), which is incorporated herein by reference. tTA was created by fusion of the tetracycline resistance operon (tet repressor) encoded in Tn10 of Escherichia coli with the activating cycline-controlled transactivator (tTA) and the tTA-dependent promoter was created by combining the tet operator sequence and a minimal promoter from the human cytomegalovirus promoter IE (hCMV-IE). When tetracycline or its derivatives are added, tTA can no longer bind its target sequence within the tTA-dependent promoter and there is no expression from the tTA-dependent promoter. This is shown in FIG. 1A and explained on page s96 of Jaisser, 2000 (Jaisser, F. (2000) ‘Inducible gene expression and gene modification in transgenic mice’, Journal of the American Society of Nephrology, 11(SUPPL. 16), pp. 95-100), which is incorporated herein by reference. The mechanism of the conformational change brought by binding of tetracycline or its derivatives to tTA is described in Orth et al., 2000 (Orth, P. et al. (2000) ‘Structural basis of gene regulation by the tetracycline inducible Tet repressor-operator system’, Nature Structural Biology, 7(3), pp. 215-219. doi: 10.1038/73324), which is incorporated herein by reference. Binding of tetracycline to TetR increases the separation of the attached DNA binding domains which abolishes the affinity of TetR for its operator DNA.

A suitable promoter induced by presence of tetracycline or its derivatives is the promoter in the tet-ON system. In the tet-ON system, a reverse tetracycline-controlled transactivator (rtTA) allows transcriptional activation of a tTA-dependent promoter in the presence of tetracycline or its derivatives as described in Gossen et al (Science 23 Jun. 1995: Vol. 268, Issue 5218, pp. 1766-1769 DOI: 10.1126/science.7792603), which is incorporated herein by reference. In the absence of tetracycline or its derivatives, tTA can no longer bind its target sequence within the tTA-dependent promoter and there is no expression from the tTA-dependent promoter. This is shown in FIG. 1B and explained on page s96 of Jaisser, 2000 (Jaisser, F. (2000) ‘Inducible gene expression and gene modification in transgenic mice’, Journal of the American Society of Nephrology, 11(SUPPL. 16), pp. 95-100), which is incorporated herein by reference.

Suitably, an improved variant of the reverse tetracycline-controlled transactivator (rtTA) is used.

Suitable improved variants are described in table 1 of Urlinger et al., 2000 (Urlinger, S. et al. (2000) ‘Exploring the sequence space for tetracycline-dependent transcriptional activators: Novel mutations yield expanded range and sensitivity’, Proceedings of the National Academy of Sciences of the United States of America, 97(14), pp. 7963-7968. doi: 10.1073/pnas.130192197), which is incorporated herein by reference. Variants rtTA-S2 and rtTA-M2 were shown to have lower basal activity in FIG. 3 of Urlinger et al., 2000, which indicates minimal background expression from the tTA-dependent promoter in the absence of tetracycline or its derivatives. Additionally rtTA-M2 showed an increased sensitivity towards tetracycline and its derivatives as shown in in FIG. 3 of Urlinger et al., 2000 and functions at 10 fold lower concentrations than rtTA. In some preferred embodiments, the improved variant of rtTA is rtTA-M2 from of Urlinger et al., 2000.

Alternative improved variants are described in Table 1 of Zhou et al., 2006 (Zhou, X. et al. (2006) ‘Optimization of the Tet-On system for regulated gene expression through viral evolution’, Gene Therapy, 13(19), pp. 1382-1390. doi: 10.1038/sj.gt.3302780), which is incorporated herein by reference. The majority of these variants were shown to have higher transcriptional activity and doxycycline sensitivity than rtTA as described in FIG. 3 of Zhou et al., 2006. The highest performing variants were seven-fold more active and 100 times more sensitive to doxycycline. In some preferred embodiments, the improved variant of rtTA is V14, V15 or V16 from Zhou et al., 2006.

Suitable commercially available tetracycline-inducible system is the T-Rex system from Life-Technologies (see e.g. Life-Technologies (2014) ‘Inducible Protein Expression Using the T-REx™ System’, 1, pp. 1-12. Available at: www.lifetechnologies.com/de/de/home/references/protocols/proteins-expression-isolation-and-analysis/protein-expression-protocol/inducible-protein-expre ssion-using-the-trex-system.reg.us.html/).

Induction via, e.g. Tetracycline Absence and Estrogen Presence—The inducible promoter may be induced by absence of a molecule and presence of a different molecule. In some embodiments, the inducible promoter may be induced by removal of tetracycline and addition of estrogen as described in lida et al., 1996 (Iida, A. et al. (1996) ‘Inducible gene expression by retrovirus-mediated transfer of a modified tetracycline-regulated system.’, Journal of virology, 70(9), pp. 6054-6059. doi: 10.1128/jvi.70.9.6054-6059.1996), which is incorporated herein by reference. This specific inducibility was achieved by the addition of the ligand-binding domain of the estrogen receptor to the carboxy terminal of the tTA transactivator. Such modified transactivator was shown result in high expression of the gene of interest in the absence of tetracycline and the presence of estrogen as shown in FIG. 3 of lida et al., 1996.

Induction via Small Molecule Enhancers—The inducible promoter may be induced by small molecule enhancers. Suitable promoters induced by small molecule enhancers such as aromatic carboxylic acids, hydroxamic acids and acetamides are described in Allen et al. (Biotechnol. Bioeng. 2008; 100: 1193-1204), which is incorporated herein by reference.

Mifepristone (RU-486)-Inducible Promoters—The inducible promoter may be induced by a synthetic steroid. In some embodiments, the inducible promoter may be induced by mifepristone, also known as RU-486. A hybrid mifespristone-responsive transcription factor, LexPR transactivator, was created by Emelyanov and Parinov, 2008 (Emelyanov, A. and Parinov, S. (2008) ‘Mifepristone-inducible LexPR system to drive and control gene expression in transgenic zebrafish’, Developmental Biology, 320(1), pp. 113-121. doi: 10.1016/j.ydbio.2008.04.042, which is incorporated herein by reference) by fusion of the DNA-binding domain of the bacterial LexA repressor, a truncated ligand-binding domain of the human progesterone receptor and the activation domain of the human NF-kB/p65 protein. Upon addition of mifepristone, LexPR induces expression from a promoter sequence harbouring LexA binding sites as shown in FIG. 1 and FIG. 2 of Emelyanov and Parinov, 2008. Suitable commercially available mifepristone-inducible system is the GeneSwitch System (see e.g., Fisher, T. (1994) ‘Inducible Protein Expression Using GeneSwitch™ Technology’, pp. 1-25).

Cumate-Inducible Promoters—In some embodiments, the inducible promoter may be induced by the presence or the absence of cumate.

In the cumate switch system from Mullick et al., 2006 (Mullick, A. et al. (2006) ‘The cumate gene-switch: A system for regulated expression in mammalian cells’, BMC Biotechnology, 6, pp. 1-18. doi: 10.1186/1472-6750-6-43, which is incorporated herein by reference), a repressor CymR blocks transcription from a promoter comprising CuO sequence placed downstream of the promoter. Once cumate is added, the CymR repressor is unable to bind to CuO and transcription from a promoter comprising CuO can proceed. This is shown in FIG. 1B and FIG. 2 from Mullick et al., 2006.

In an alternative cumate switch system, a chimeric transactivator (cTA) created from the fusion of CymR with the activation domain of VP16 does not prevent transcription from a promoter comprising CuO sequence upstream of a promoter in the presence of cumate. In the absence of cumate, the chimeric transactivator (cTA) binds to the CuO sequence and prevents transcription. This is shown in FIG. 1C and FIG. 3 from Mullick et al., 2006.

In a third configuration, a reverse chimeric transactivator (rcTA) prevents transcription from a promoter comprising CuO sequence upstream of a promoter in the absence of cumate. In the presence of cumate, the rcTA binds to the CuO sequence and transcription from the promoter comprising CuO sequence can proceed. This is shown in FIG. 1D and FIG. 7 from Mullick et al., 2006.

Suitable commercially available cumate-inducible systems is found from SBI Biosciences (see SBI (2020) ‘Cumate-inducible Systems For the ultimate in gene expression control, use SBI's cumate—CUMATE-INDUCIBLE SYSTEMS’, pp. 1-13, which is incorporated herein by reference).

4-hydroxytamoxifen (OHT)-Inducible Promoters—The inducible promoter may be induced by 4-hydroxytamoxifen (OHT). Suitable 4-hydroxytamoxifen inducible promoters are described by Feil et al. (Biochemical and Biophysical Research Communications Volume 237, Issue 3, 28 Aug. 1997, Pages 752), which is incorporated herein by reference.

Gas-Inducible Promoters—The inducible promoter may be a gas-inducible promoter, e.g. acetaldehyde-inducible. Suitable gas-inducible promoters are described in Weber et al., 2004 (Weber, W. et al. (2004) ‘Gas-inducible transgene expression in mammalian cells and mice’, Nature Biotechnology, 22(11), pp. 1440-1444. doi: 10.1038/nbt1021), which is incorporated herein by reference. The native acetaldehyde-inducible AlcR-PalcA system from Aspergillus nidulans has been adapted for mammalian use by introducing an AlcR-specific operator module to a human minimal promoter, together called P_{AIR}, as shown in FIG. 1A. When AlcR is constitutively expressed in the cell of interest, upon introduction of acetaldehyde, acetaldehyde binds to AlcR and, in turn, the gene of interest which is under the control of the PAIR promoter is expressed, as shown in FIG. 1C, FIG. 2 and FIG. 3. In the absence of acetaldehyde, there is no expression of the gene of interest.

Riboswitch, Ribozyme and Aptazyme-Inducible Promoters—The inducible promoter may be induced by the presence or absence of a ribozyme. The ribozyme can, in turn be, be induced by a ligand.

The inducible promoter may be induced in the absence of a metabolite. In some embodiments, the metabolite may be glucosamine-6-phosphate-responsive. Suitable ribozyme which acts as a glucosamine-6-phosphate-responsive gene repressor is described by Winkler et al., 2004 (Winkler, W. C. et al. (2004) ‘Control of gene expression by a natural metabolite-responsive ribozyme’, Nature, 428(6980), pp. 281-286. doi: 10.1038/nature02362), which is incorporated herein by reference. The ribozyme is activated by glucosamine-6-phosphate in a concentration dependent manner as shown in FIG. 2C and cleaves the messenger RNA of the glmS gene. Upon modification, it is possible that this natural system may be applied to control of a gene of interest other than the glmS gene.

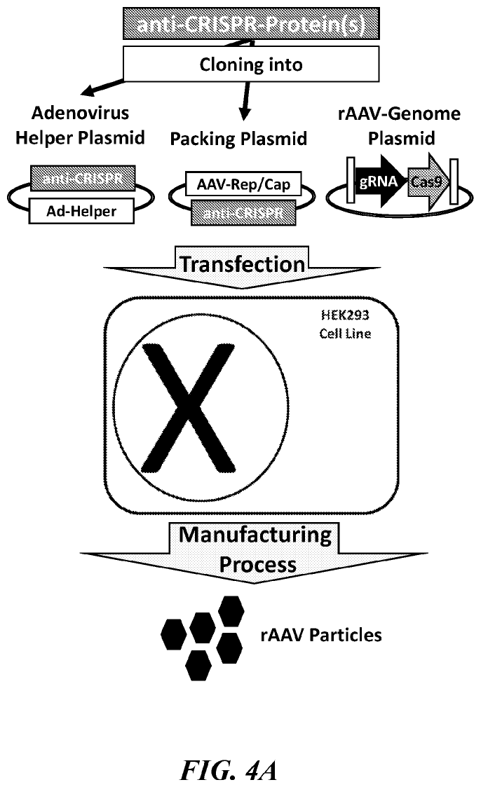

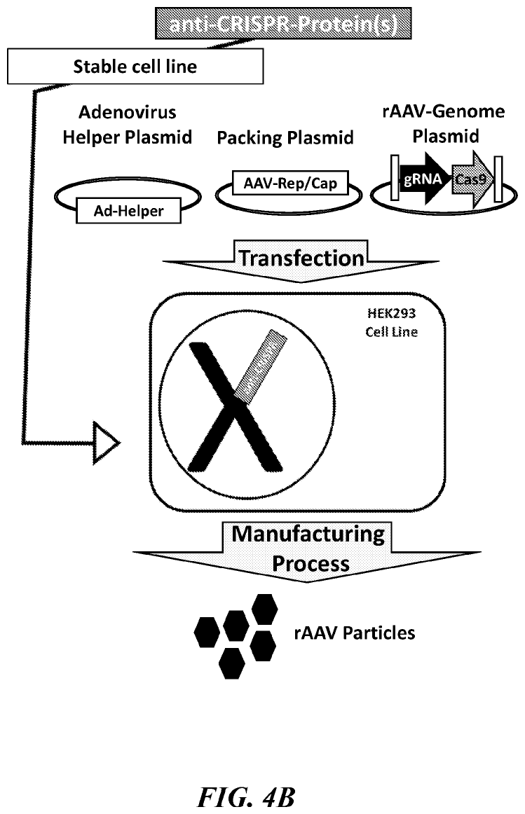

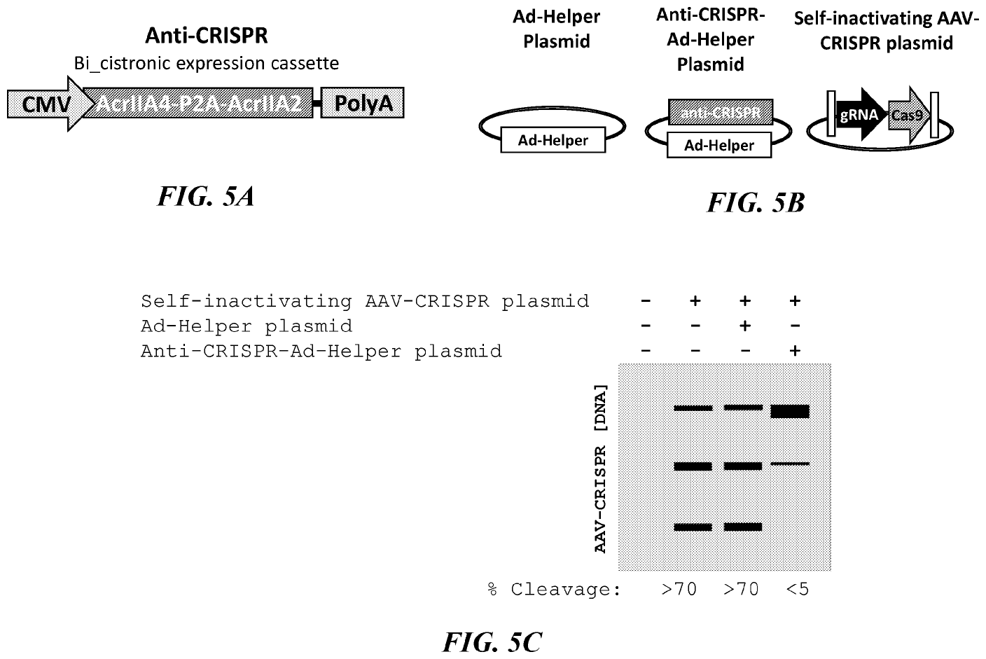

Protein expression can also be downregulated by ligand-inducible aptazyme. Protein expression can be downregulated by aptazyme which downregulate protein expression by small molecule-induced self-cleavage of the ribozyme resulting in mRNA degradation Zhong et al., 2016 (Zhong, G. et al. (2016) ‘Rational design of aptazyme riboswitches for efficient control of gene expression in mammalian cells’, eLife, 5(November 2016). doi: 10.7554/eLife.18858), which is incorporated herein by reference. Suitable aptazymes are shown in FIG. 4A of (Zhong et al., 2016). These aptazymes reduce relative expression of a gene of interest as shown in FIG. 4 of (Zhong et al., 2016).

Protein expression can also be upregulated by a small-molecule dependent ribozyme. The ribozyme may be tetracycline-dependent. Suitable tetracycline-dependent ribozymes which can switch on protein expression by preventing ribozyme cleavage which otherwise cleaves mRNA in the absence of ligand is described in Beilstein et al. (ACS Synth. Biol. 2015, 4, 5, 526-534), which is incorporated herein by reference.

Protein expression can also be regulated by a guanine dependent aptazyme as described by Nomura et al. (Chem. Commun., 2012,48, 7215-7217) which is incorporated herein by reference.

Additionally, an RNA architecture that combines a drug-inducible allosteric ribozyme with a microRNA precursor analogue that allows chemical induction of RNAi in mammalian cells is described in Kumar et al (J. Am. Chem. Soc. 2009, 131, 39, 13906-13907), which is incorporated herein by reference.

Metallothionein-Inducible Promoters—Metallothionein-inducible promoters have been described in the literature. See for example Shinichiro Takahashi “Positive and negative regulators of the metallothionein gene” Molecular Medicine Reports Mar. 9, 2015, P795-799, which is incorporated herein by reference.

Rapamycin-Inducible Promoters—The inducible promoter may be induced by a small molecule drug such as rapamycin. A humanized system for pharmacologic control of gene expression using rapamycin is described in Rivera et al., 1996 (Rivera et al Nature Medicine volume 2, pages 1028-1032(1996)), which is incorporated herein by reference. The natural ability of rapamycin to bind to FKBP12 and, in turn, for this complex to bind to FRAP was used by Rivera et al., 1996 to induce rapamycin-specific expression of a gene of interest. This was achieved by fusing one of the FKBP12/FRAP proteins to a DNA binding domain and the other protein to an activator domain. If the FKBP is fused with a DNA binding domain and FRAP is fused to an activator domain, there would be no transcription of the gene of interest in the absence of rapamycin since FKBP and FRAP do not interact, as shown in FIG. 1b. In the presence of rapamycin, FKBP and FRAP interact and the DNA binding domain and the activator domain are brought into close contact, resulting in transcription of the gene of interest as shown in FIG. 2 and FIG. 3.

Chemically-Induced Proximity-Inducible Promoters—The inducible promoter may be controlled by the chemically induced proximity. Suitable small molecule-based systems for controlling protein abundance or activities is described in Liang et al. (Sci Signal. 2011 Mar. 15; 4(164):rs2. doi: 10.1126/scisignal.2001449), which is incorporated herein by reference.

Gene expression may be induced by chemically induced proximity by a molecule combining two protein binding surfaces as shown in Belshaw et al., 1996 (Belshaw, P. J. et al. (1996) ‘Controlling protein association and subcellular localization with a synthetic ligand that induces heterodimerization of proteins’, Proceedings of the National Academy of Sciences of the United States of America, 93(10), pp. 4604-4607), which is incorporated herein by reference. Transcriptional activation of a gene of interest by chemically induced proximity by a molecule combining two protein binding surfaces is shown in FIG. 3 of Belshaw et al.

Rheoswitch® Inducible Promoters—The inducible promoter may be induced by small synthetic molecules. In some embodiments, these small synthetic molecules may be diacylhydrazine ligands. Suitable systems for inducible up- and down-regulation of gene expression is described in Cress et al. (Volume 66, Issue 8 Supplement, pp. 27) or Barrett et al. (Cancer Gene Therapy volume 25, pages 106-116(2018)), which are incorporated herein by reference. The RheoSwitch® system consists of two chimeric proteins derived from the ecdysone receptor (EcR) and RXR that are fused to a DNA-binding domain and an acidic transcriptional activation domain, respectively. The nuclear receptors can heterodimerize to create a functional transcription factor upon binding of a small molecule synthetic ligand and activate transcription from a responsive promoter linked to a gene of interest.

CRISPR-Inducible Promoters—Gene expression may be induced by a on CRISPR-based transcription regulators. A nuclease-deficient Cas9 can be directed to a sequence of interest by designing its associated single guide RNA (sgRNA) and it can modulate the gene expression by tethering of effector domains on the sgRNA-Cas9 complex as shown in FIG. 1A of Ferry, Lyutova and Fulga, 2017 (Ferry, Q. R. V., Lyutova, R. and Fulga, T. A. (2017) ‘Rational design of inducible CRISPR guide RNAs for de novo assembly of transcriptional programs’, Nature Communications. Nature Publishing Group, 8, pp. 1-10. doi: 10.1038/ncomms14633), which is incorporated herein by reference. Suitable versatile inducible-CRISPR-TR platform based on minimal engineering of the sgRNA is described in Ferry, Lyutova and Fulga, 2017.

The CRISPR-based transcriptional regulation may in turn be induced by drugs. Suitable drug inducible CRISPR-based transcription regulators systems are shown in Zhang et al., 2019 (Zhang, J. et al. (2019) ‘Drug Inducible CRISPR/Cas Systems’, Computational and Structural Biotechnology Journal. Elsevier B. V., 17, pp. 1171-1177. doi: 10.1016/j.csbj.2019.07.015), which is incorporated herein by reference.

In one embodiment, contacting the cell with an inducer or applying a suitable inducing condition to the cell results in expression of the gene operatively linked to the inducible promoter.

Inducible promoters described herein can further control expression of an inducer or repressor of an inducible promoter, e.g., an inducer or repressor of a second, different promoter, or an inducer or repressor of itself. In one embodiment, the cell comprises a first inducible promoter that is operatively linked to a repressible element that can stop protein expression.

In one embodiment, the first inducible promoter that further encodes a protein that represses expression of the first inducible promoter.

In one embodiment, the cell comprises a first inducible promoter that further encodes a protein that induces expression of a second inducible promoter.

Tissue-Specific Promoters

In some embodiments disclosed herein, the promoter is a tissue-specific promoter. In some embodiments disclosed herein, the tissue-specific promoter is a liver specific promoter, and can be selected from promoters including, but not limited to, those disclosed in Table 3. Liver-specific” or “liver-specific expression” refers to the ability of a cis-regulatory element, cis-regulatory module or promoter to enhance or drive expression of a gene in the liver (or in liver-derived cells) in a preferential or predominant manner as compared to other tissues (e.g. spleen, muscle, heart, lung, and brain). Expression of the gene can be in the form of mRNA or protein. In preferred embodiments, liver-specific expression is such that there is negligible expression in other (i.e. non-liver) tissues or cells, i.e. expression is highly liver-specific.

A liver-specific promoter includes a liver-specific cis-regulatory element (CRE), a synthetic liver-specific cis-regulatory module (CRM) or a synthetic liver-specific promoter as disclosed herein, in Table 3. These liver-specific promoter elements include minimal liver-specific promoters.

Liver-specific promoter elements are further described in, e.g., International Application No. PCT/GB2019/053267, which is incorporated herein by reference in its entirety.

Table 3 shows exemplary liver-specific promoter sequences. The relatively small size of liver-specific promote sequence in Table 3 is advantageous because it takes up the minimal amount of the payload of the vector. This is particularly important when a CRE is used in a vector with limited capacity, such as an AAV-based vector.

TABLE 3
-Liver-specific promoters
(These are liver-specific promoters
comprising cis-regulatory modules (CRMs)):

| Liver-specific | |
|---|---|
| CRM Promoter | SEQUENCE |
| CRM_SP0131 | GGCCCGGGAGGCGCCCTTTGGACCTTTTGCAA |
| (CRM_LVR_131) | TCCTGGCGCACTGAACCCTTGACCCCTGCCCT |
| | GCAGCCCCCGCAGCTTGCTGTTTGCCCACTCT |
| | ATTTGCCCAGCCCCAGCCCTGGAGAGTCCTTT |
| | AGCAGGGCAAAGTGCAACATAGGCAGACCTTA |
| | AGGGATGACTCAGTAACAGATAAGCTTTGTGT |
| | GCCTGCA (SEQ ID NO: 9) |
| CRM_SP0239 | CAGGCTTTCACTTTCTCGCCAACTTACAAGGC |
| | CTTTCTGTGTAAACAATACCTGAACCTTTACC |
| | CCGTTGCCCGGCAACGGCCAGGTCTGTGCCAA |
| | GTGTTTGAGGTTAATTTTTAAAAAGCAGTCAA |
| | AAGTCCAAGTGGCCCTTGGCAGCATTTACTCT |
| | CTCTGTTTGCTCTGGTTAATAATCTCAGGAGC |
| | ACAAACATTCCGGCCCGGGAGGCGCCCTTTGG |
| | ACCTTTTGCAATCCTGGCGCACTGAACCCTTG |
| | ACCCCTGCCCTGCAGCCCCCGCAGCTTGCTGT |
| | TTGCCCACTCTATTTGCCCAGCCCCAGCCCTG |
| | GAGAGTCCTTTAGCAGGGCAAAGTGCAACATA |
| | GGCAGACCTTAAGGGATGACTCAGTAACAGAT |
| | AAGCTTTGTGTGCCTGCA |
| | (SEQ ID NO: 10) |
| CRM_SP0240 | CAGGCTTTCACTTTCTCGCCAACTTACAAGGC |
| | CTTTCTGTGTAAACAATACCTGAACCTTTACC |
| | CCGTTGCCCGGCAACGGCCAGGTCTGTGCCAA |
| | GTGTTTG (SEQ ID NO: 11) |
| CRM_SP0246 | AGGTTAATTTTTAAAAAGCAGTCAAAAGTCCA |
| | AGTGGCCCTTGGCAGCATTTACTCTCTCTGTT |
| | TGCTCTGGTTAATAATCTCAGGAGCACAAACA |
| | TTCCGGCCCGGGAGGCGCCCTTTGGACCTTTT |
| | GCAATCCTGGCGCACTGAACCCTTGACCCCTG |
| | CCCTGCAGCCCCCGCAGCTTGCTGTTTGCCCA |
| | CTCTATTTGCCCAGCCCCAG |
| | (SEQ ID NO: 12) |
| CRM_SP0265 | AGGTTAATTTTTAAAAAGCAGTCAAAAGTCCA |
| (CRM_LVR_131_A1) | AGTGGCCCTTGGCAGCATTTACTCTCTCTGTT |
| | TGCTCTGGTTAATAATCTCAGGAGCACAAACA |
| | TTCCTGTACCGGCCCGGGAGGCGCCCTTTGGA |
| | CCTTTTGCAATCCTGGCGCACTGAACCCTTGA |
| | CCCCTGCCCTGCAGCCCCCGCAGCTTGCTGTT |
| | TGCCCACTCTATTTGCCCAGCCCCAGCCCTGG |
| | AGAGTCCTTTAGCAGGGCAAAGTGCAACATAG |
| | GCAGACCTTAAGGGATGACTCAGTAACAGATA |
| | AGCTTTGTGTGCCTGCA |
| | (SEQ ID NO: 13) |
| CRM_SP0412 | AGGTTAATTTTTAAAAAGCAGTCAAAAGTCCA |
| | AGTGGCCCTTGGCAGCATTTACTCTCTCTGTT |
| | TGCTCTGGTTAATAATCTCAGGAGCACAAACA |
| | TTCCCCCTGTTCAAACATGTCCTAATACTCTG |
| | TCTCTGCAAGGGTCATCAGTAGTTTTCCATCT |
| | TACTCAACATCCTCCCAGTG |
| | (SEQ ID NO: 14) |

Other liver specific promoters include, but are not limited to promoters for the LDL receptor, Factor VIII, Factor IX, phenylalanine hydroxylase (PAH), ornithine transcarbamylase (OTC), and α1-antitrypsin (hAAT), and HCB promoter. Other liver specific promoters include the AFP (alpha fetal protein) gene promoter and the albumin gene promoter, as disclosed in EP Patent Publication 0 415 731, the α-1 antitrypsin gene promoter, as disclosed in Rettenger, Proc. Natl. Acad. Sci. 91 (1994) 1460-1464, the fibrinogen gene promoter, the APO-A1 (Apolipoprotein A1) gene promoter, and the promoter genes for liver transference enzymes such as, for example, SGOT, SGPT and γ-glutamyle transferase. See also 2001/0051611 and PCT Patent Publications WO 90/07936 and WO 91/02805, which are incorporated herein in their entirety by reference. In some embodiments, the liver specific promoter is a recombinant liver specific promoter, e.g., as disclosed in US20170326256A1, which is incorporated herein in its entirety by reference.

In some embodiments, a liver specific promoter is the hepatitis B X-gene promoter and the hepatitis B core protein promoter. In some embodiments, liver specific promoters can be used with their respective enhancers. The enhancer element can be linked at either the 5′ or the 3′ end of the nucleic acid encoding the lysosomal enzyme. The hepatitis B X gene promoter and its enhancer can be obtained from the viral genome as a 332 base pair EcoRV-NcoI DNA fragment employing the methods described in Twu, J Virol. 61 (1987) 3448-3453. The hepatitis B core protein promoter can be obtained from the viral genome as a 584 base pair BamHI-BgIII DNA fragment employing the methods described in Gerlach, Virol 189 (1992) 59-66. It may be necessary to remove the negative regulatory sequence in the BamHI-BgIII fragment prior to inserting it.

Functional Variants of Liver-Specific Promoters

In some embodiments, a functional variant of a liver-specific promoter can be viewed as a promoter element which, when substituted in place of a reference promoter element in a promoter, substantially retains its activity. For example, a functional variant of liver-specific promoter which comprises a functional variant of a given promoter disclosed in Table 3 preferably retains at least 35%, or at least 40%, or at least 45%, or at least 50%, or at least 55%, or at least 60%, or at least 70% or at least 80% of its activity, more preferably at least 90% of its activity, more preferably at least 95% of the activity of the unchanged promoter, and yet more preferably 100% of the activity (as compared to the unchanged promoter sequence comprising the unmodified promoter element).

In addition, one or more promoters, which can be the same or different, can be present in the same nucleic acid molecule, either together or positioned at different locations on the nucleic acid molecule. Furthermore, an internal ribosome entry signal (IRES) and/or other ribosome-readthrough element can be present on the nucleic acid molecule. One or more such IRESs and/or ribosome readthrough elements, which can be the same or different, can be present in the same nucleic acid molecule, either together and/or at different locations on the nucleic acid molecule. Such IRESs and ribosome readthrough elements can be used to translate messenger RNA sequences via cap-independent mechanisms when multiple nuclease sequences are present on a nucleic acid molecule.

The components of the gene-editing system can be present in a vector and such a vector can be present in a cell. Any suitable vector is encompassed in the embodiments of this invention, including, but not limited to, nonviral vectors (e.g., nucleic acids, minicircles, linear DNA, plasmids, poloxymers, exosomes, and liposomes), viral vectors and synthetic biological nanoparticles (BNP) (e.g., synthetically designed from different adeno-associated viruses, as well as other parvoviruses).

### Cas Protein

In one embodiment, the gene-editing protein is a CRISPR-associated nuclease, for example a Cas protein. Exemplary Cas proteins include, but are not limited to, Cpf1, C2c1, C2c3, Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, C2c1, C2c3, Cas12a, Cas12b, Cas12c, Cas12d, Cas12e, Cas13a, Cas13b, and Cas13c.

In one embodiment, the Cas protein is Cas9 or Cas9 variant, e.g. isolated from the bacterium Streptococcus pyogenes (SpCas9). The CRISPR-associate nuclease associates with guide RNA (gRNA) that guides the nuclease to the desired target sequence, e.g. having a protospacer adjacent motif (PAM) sequence, downstream of the target sequence for its cutting action. Once Cas9 recognizes the PAM sequence (5′-NGG-3 in case of SpCas9, where N is any nucleotide), it creates a double-strand break (DSB) at the target locus. Cas9 activity is a collective effort of two parts of the protein: the recognition lobe that senses the complementary sequence of gRNA and the nuclease lobe that cleaves the DNA.

In one embodiment, the Cas protein is an enhanced specificity spCas9 (eSpCas9) variant. eSpCas9 variants are further described in Slaymaker, et al. Science. 2016; 351(6268): 84-88, which is incorporated herein by reference in its entirety.

In one embodiment the Cas protein is a natural variant of Cas. Cas9 Variants include e.g. Staphylococcus aureus (SaCas9), Streptococcus thermophilus (StCas9), Neisseria meningitidis (NmCas9), Francisella novicida (FnCas9), and Campylobacter jejuni (CjCas9), to name a few, in CRISPR experiments. The nuclease can be determined based on preferred PAM sequence or size, e.g. the SaCas9 nuclease is about 1 kb smaller in size than SpCas9 so it can be packaged into viral vectors more easily; and e.g. CasX and CasY (Burstein, David, et al. New CRISPR-Cas systems from uncultivated microbes. Nature 542.7640 (2017): 237, incorporated by reference in its entirety) are two of the most compact naturally occurring CRISPR variants.

Sequences for Cas9 for various species are known in the art. For example, S. aureus Cas9 (saCas9) has the sequence of SEQ ID NO: 3.

SEQ ID NO: 3 is an amino acid sequence encoding S. aureus Cas9.

(SEQ ID NO: 3)
MKRNYILGLD IGITSVGYGI IDYETRDVID AGVRLFKEAN
VENNEGRRSK RGARRLKRRR RHRIQRVKKL LFDYNLLTDH
SELSGINPYE ARVKGLSQKL SEEEFSAALL HLAKRRGVHN
VNEVEEDTGN ELSTKEQISR NSKALEEKYV AELQLERLKK
DGEVRGSINR FKTSDYVKEA KQLLKVQKAY HQLDQSFIDT
YIDLLETRRT YYEGPGEGSP FGWKDIKEWY EMLMGHCTYF
PEELRSVKYA YNADLYNALN DLNNLVITRD ENEKLEYYEK
FQIIENVFKQ KKKPTLKQIA KEILVNEEDI KGYRVTSTGK
PEFTNLKVYH DIKDITARKE IIENAELLDQ IAKILTIYQS
SEDIQEELTN LNSELTQEEI EQISNLKGYT GTHNLSLKAI
NLILDELWHT NDNQIAIFNR LKLVPKKVDL SQQKEIPTTL
VDDFILSPVV KRSFIQSIKV INAIIKKYGL PNDIIIELAR
EKNSKDAQKM INEMQKRNRQ TNERIEEIIR TTGKENAKYL
IEKIKLHDMQ EGKCLYSLEA IPLEDLLNNP FNYEVDHIIP
RSVSFDNSFN NKVLVKQEEN SKKGNRTPFQ YLSSSDSKIS
YETFKKHILN LAKGKGRISK TKKEYLLEER DINRFSVQKD
FINRNLVDTR YATRGLMNLL RSYFRVNNLD VKVKSINGGF
TSFLRRKWKF KKERNKGYKH HAEDALIIAN ADFIFKEWKK
LDKAKKVMEN QMFEEKQAES MPEIETEQEY KEIFITPHQI
KHIKDFKDYK YSHRVDKKPN RELINDTLYS TRKDDKGNTL
IVNNLNGLYD KDNDKLKKLI NKSPEKLLMY HHDPQTYQKL
KLIMEQYGDE KNPLYKYYEE TGNYLTKYSK KDNGPVIKKI
KYYGNKLNAH LDITDDYPNS RNKVVKLSLK PYRFDVYLDN
GVYKFVTVKN LDVIKKENYY EVNSKCYEEA KKLKKISNQA
EFIASFYNND LIKINGELYR VIGVNNDLLN RIEVNMIDIT
YREYLENMND KRPPRIIKTI ASKTQSIKKY STDILGNLYE
VKSKKHPQII KKG

In one embodiment, the Cas protein is a Cas 9 derived from Campylobacter jejuni (C. jejuni). This C. jejuni Cas9 (CjCas9) is further described in, e.g., International patent application WO2016021973A1, which is incorporated herein by reference in its entirety.

SEQ ID NO: 4 is an amino acid sequence encoding CjCas9.

(SEQ ID NO: 4)
MARILAFDIG ISSIGWAFSE NDELKDCGVR IFTEVENPKT
GESLALPRRL ARSARKRLAR RKARLNHLKH LIANEFKLNY
EDYQSFDESL AKAYKGSLIS PYELRFRALN ELLSKQDFAR
VILHIAKRRG YDDIKNSDDK EKGAILKAIK QNEEKLANYQ
SVGEYLYKEY FQKFKENSKE FTNVRNKKES YERCIAQSFL
KDELKLIFKK QREFGFSFSK KFEEEVLSVA FYKRALKDFS
HLVGNCSFFT DEKRAPKNSP LAFMFVALTR IINLLNNLKN
TEGILYTKDD LNALLNEVLK NGTLTYKQTK KLLGLSDDYE
FKGEKGTYFI EFKKYKEFIK ALGEHNLSQD DLNEIAKDIT
LIKDEIKLKK ALAKYDLNQN QIDSLSKLEF KDHLNISFKA
LKLVTPLMLE GKKYDEACNE LNLKVAINED KKDFLPAFNE
TYYKDEVTNP VVLRAIKEYR KVLNALLKKY GKVHKINIEL
AREVGKNHSQ RAKIEKEQNE NYKAKKDAEL ECEKLGLKIN
SKNILKLRLF KEQKEFCAYS GEKIKISDLQ DEKMLEIDHI
YPYSRSFDDS YMNKVLVFTK QNQEKLNQTP FEAFGNDSAK
WQKIEVLAKN LPTKKQKRIL DKNYKDKEQK NFKDRNLNDT
RYIARLVLNY TKDYLDFLPL SDDENTKLND TQKGSKVHVE
AKSGMLTSAL RHTWGFSAKD RNNHLHHAID AVIIAYANNS
IVKAFSDFKK EQESNSAELY AKKISELDYK NKRKFFEPFS
GFRQKVLDKI DEIFVSKPER KKPSGALHEE TFRKEEEFYQ
SYGGKEGVLK ALELGKIRKV NGKIVKNGDM FRVDIFKHKK
TNKFYAVPIY TMDFALKVLP NKAVARSKKG EIKDWILMDE
NYEFCFSLYK DSLILIQTKD MQEPEFVYYN AFTSSTVSLI
VSKHDNKFET LSKNQKILFK NANEKEVIAK SIGIQNLKVF
EKYIVSALGE VTKAEFRQRE DFKK

In one embodiment the Cas protein is Cas12a (also known as Cpf1). As Cas9 requires guanine-rich PAM sequence of NGG, it is not well suited for targeting AT-rich sequences. Zetsche et al. characterized a nuclease (see e.g. US Patent Application US 2016/0208243 for sequence and variants, incorporated by reference in its entirety), CRISPR from Prevotella and Francisella 1 (Cpf1; now classified as Cas12a) can be used when targeting AT-rich DNA sequences. Cpf1 creates a staggered double-stranded cut, rather than blunt-end cut generated by SpCas9, in the target DNA, and is useful for experiments relying on the HDR repair outcome. Also, Cpf1 is smaller than SpCas9 and does not require a tracer RNA. The guide RNA required by Cpf1 is therefore shorter in length, making it more economical to produce.

Sequences for Cpf1 for various species are known in the art. For example, Acidaminococcus sp. Cpf1 has the sequence of SEQ ID NO: 5.

SEQ ID NO: 5 is an amino acid sequence encoding Acidaminococcus sp. Cpf1.

(SEQ ID NO: 5)
MTQFEGFTNLYQVSKTLRFELIPQGKTLKHIQEQGFIEEDKARNDHYKEL
KPIIDRIYKTYADQCLQLVQLDWENLSAAIDSYRKEKTEETRNALIEEQA
TYRNAIHDYFIGRTDNLTDAINKRHAEIYKGLFKAELFNGKVLKQLGTVT
TTEHENALLRSFDKFTTYFSGFYENRKNVFSAEDISTAIPHRIVQDNFPK
FKENCHIFTRLITAVPSLREHFENVKKAIGIFVSTSIEEVFSFPFYNQLL
TQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAE
ALFNELNSIDLTHIFISHKKLETISSALCDHWDTLRNALYERRISELTGK
ITKSAKEKVQRSLKHEDINLQEIISAAGKELSEAFKQKTSEILSHAHAAL
DQPLPTTLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGERNLIYITVIDSTGKILEQRSLNTIQQFDYQKKLDNREKE
RVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGFMPAWDIVFEKNETQFDAK
GTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFD
SRFQNPEWPMDADANGAYHIALKGQLLLNHLKESKDLKLQNGISNQDWLA
YIQELRN

In one embodiment, the Cas protein is an engineered Cas9 Variant, e.g. a Cas9 Nickase, or a dead Cas9 for use in CRISPRi or CRISPRa systems. For example, variants that nick a single DNA strand instead of creating a double-strand break. (See e.g. Cong, Le, et al. Multiplex genome engineering using CRISPR/Cas systems. Science (2013): 1231143; Mali, Prashant, et al. CAS9 transcriptional activators for target specificity screening and paired nickases for cooperative genome engineering. Nature biotechnology 31.9 (2013): 833; Ran, F. Ann, et al. Double nicking by RNA-guided CRISPR Cas9 for enhanced genome editing specificity. Cell 154.6 (2013): 1380-1389; Cho, Seung Woo, et al. Analysis of off-target effects of CRISPR/Cas-derived RNA-guided endonucleases and nickases. Genome research 24.1 (2014): 132-141, each of which incorporated by reference in their entirety). In some embodiments two guide RNAs are used with the nCAS9. Alternatively, eSpCas9 that uses a single gRNA can be used. Although nickases show high specificity, they rely on two guide RNAs to reach the target sites, thereby reducing the number of potential target sites in the genome. An alternative was created by engineering versions of Cas9 that improved fidelity using a single guide RNA; (see e.g. Qi, Lei S., et al. Repurposing CRISPR as an RNA-guided platform for sequence-specific control of gene expression.” Cell 152.5 (2013): 1173-1183, incorporated by reference in its entirety).

In one embodiment the Cas protein is SpCas9-HF1 or HypaCas9Kleinstiver (See e.g. Benjamin P., et al. High-fidelity CRISPR-Cas9 nucleases with no detectable genome-wide off-target effects.” Nature 529.7587 (2016): 490; Chen, Janice S., et al. Enhanced proofreading governs CRISPR-Cas9 targeting accuracy. Nature 550.7676 (2017): 407, each of which are incorporated by reference in their entirety).

In one embodiment, the Cas protein is the xCas9 nuclease which recognizes a broad range of PAM sequences, increasing the target sites to 1 in 4 in the genome, (See e.g. Hu, Johnny H., et al. Evolved Cas9 variants with broad PAM compatibility and high DNA specificity. Nature (2018), incorporated by reference in its entirety).

In one embodiment, the Cas protein is a split Cas9 fusions with fluorescent proteins, e.g., GFP. This would allow imaging of genomic loci (see “Dynamic Imaging of Genomic Loci in Living Human Cells by an Optimized CRISPR/Cas System” Chen B et al. Cell 2013), but in an inducible manner. As such, in some embodiments, one or more of the Cas9 parts may be associated (and in particular fused with) a fluorescent protein, for example GFP. In general, any use that can be made of a Cas9, whether wt, nickase or a dead-Cas9 (with or without associated functional domains) can be pursued using the split Cas9 approach.

In one embodiment, the Cas protein is a dimeric CRISPR RNA-guided Fokl nuclease (see., e.g., Tsai S G, et al. Nat Biotechnol. 2014. 32(6):569-576, which is incorporated herein by reference in its entirety).

In one embodiment the Cas protein is an inactive Cas9, Dead Cas9 (also referred to as dCAS9). The dead Cas9 (dCas9) CRISPR variant is made by simply inactivating the catalytic nuclease domains while maintaining the recognition domains that allow guide RNA-mediated targeting to specific DNA sequences (Komor, Alexis C., et al. Programmable editing of a target base in genomic DNA without double-stranded DNA cleavage. Nature 533.7603 (2016): 420, incorporated by reference in its entirety). dCas9 is known to silence gene expression by physically blocking the transcription. dCas9 has also been fused to other proteins and used in various applications. For instance, gene activators or inhibitors can be fused to the dCas9 to activate or repress gene expression (CRISPRa and CRISPRi). Also, tagging a fluorescent dye to the dCas9 has enabled visualization of specific DNA fragments the genome (Gaudelli, Nicole M., et al. Programmable base editing of A⋅T to G⋅C in genomic DNA without DNA cleavage Nature 551.7681 (2017): 464, incorporated by reference in its entirety). In one embodiment, FokI fused dCas9 is used (Abudayyeh, Omar O., et al. C2c2 is a single-component programmable RNA-guided RNA-targeting CRISPR effector Science 353.6299 (2016): aaf557314, incorporated by reference in its entirety).

In one embodiment, the inactivated Cas protein is a functional gene-editing nuclease by serving as a base editor. Base editor enzymes consist of a dead Cas9 domain fused with catalytic enzyme cytidine aminase that converts GC to AT or for example, a tRNA adenosine deaminase fused with Cas9 to convert AT to GC, thus allowing for a complete range of nucleotide exchanges in the genome: See e.g. Komor, Alexis C., et al. Programmable editing of a target base in genomic DNA without double-stranded DNA cleavage Nature 533.7603 (2016): 420; Gaudelli, Nicole M., et al. Programmable base editing of A⋅ T to G⋅ C in genomic DNA without DNA cleavage.” Nature 551.7681 (2017): 464; incorporated by reference in their entirety).

High dosage of a nuclease, for example, Cas9 can exacerbate indel frequencies at off-target sequences which exhibit few mismatches to the guide strand. Such sequences are especially susceptible, if mismatches are non-consecutive and/or outside of the seed region of the guide. Herein, we describe a means to mitigate the off-target effects, by specific regulation of nuclease activity, both temporal control and local control of CRISPR associated nuclease activity. The gene-editing system described herein, can be used to reduce dosage in long-term expression experiments and therefore result in reduced off-target indels compared to constitutively active CRISPR associated nuclease, e.g. Cas9. In some embodiments, additional methods to minimize the level of toxicity and off-target effect are used and include for example, use of Cas nickase mRNA (for example S. pyogenes Cas9 with the D10A mutation) and a pair of guide RNAs targeting a site of interest, See also WO 2014/093622 (PCT/US2013/074667) herein incorporated by reference in its entirety.

Physical transduction techniques can also be used, such as liposome delivery and receptor-mediated and other endocytosis mechanisms (see, for example, Schwartzenberger et al., Blood 87:472-478, 1996). This invention can be used in conjunction with any of these and/or other commonly used nucleic acid transfer methods. Appropriate means for transfection, including viral vectors, chemical transfectants, or physico-mechanical methods such as electroporation and direct diffusion of DNA, are described by, for example, Wolff et al., Science 247:1465-1468, (1990); and Wolff, Nature 352:815-818, (1991).

Another aspect of this invention provides a cell line for expressing vectors containing a gene for a gene-editing protein with an inhibitor of the gene-editing protein to prevent leaky expression of the gene-editing protein, comprising constitutive expression of an inhibitor for the gene-editing protein. A cell, or population thereof, comprising the inhibitor of the gene-editing protein of this invention can be any eukaryotic cell including but not limited to cells from muscle (e.g., smooth muscle, skeletal muscle, cardiac muscle myocytes), liver (e.g., hepatocytes), heart, brain (e.g., neurons), eye (e.g., retinal; corneal), pancreas, kidney, endothelium, epithelium, stein cells (e.g., bone marrow; cord blood), tissue culture cells (e.g., HeLa cells) etc., as are well known in the art. The cell can be a prokaryotic cell.

In one embodiment, the cell further expresses a gene-editing protein. The expression of the gene-editing protein can be transient or constitutive.

In some embodiments, the inhibitor of the gene-editing protein of the present invention reduces the level of “leakiness” of the gene-editing protein expressed in a virus as compared with a gene-editing protein expressing-virus generated using a similar method, but without the inhibitor. By “leakiness” is meant an amount of gene-editing activity, e.g., Cas nuclease activity, that occurs without proper activation of the gene-editing system. For example, Cas nuclease activity detected in an edited cell that results in aberrant DNA cleavage at sequences other than the target sequence. The degree to which leakiness is reduced in the present system in comparison to other systems can be 5, 10, 15, 20, 25, 30, 35, 40, 45, 50, 55, 60, 65, 70, 75, 80, 85, 90, 95, or 100% less than the amount of leakiness observed in art-known systems (e.g., methods that do not utilize the inhibitor of the gene-editing protein). As one example, the amount of leakiness of a gene-editing protein (e.g., a Cas protein) can be determined by employing whole genome sequencing in a cell expressing a gene-editing protein and an inhibitor of the gene-editing protein and a cell only expressing a gene-editing protein. One would expect to find a higher incidence of mutations in a cell that has aberrant gene-editing activity (e.g., leakiness of the gene-editing protein). Further, assays for broadly detecting nuclease activity are known in the art. For example, a CRISPRuTest™ Functional Cas9 Activity Assay can be used to measure Cas9 nuclease activity in any mammalian cell system. The CRISPRuTest Kit provides a FACS-based assay to assess the Cas9 nuclease activity in cells expressing Streptococcus pyogenes Cas9. This kit contains pseudoviral packaged lentiviral constructs with a green fluorescent protein (GFP) gene whose fluorescence is disrupted in cells with active Cas9 nuclease activity. The inhibitor of the gene-editing protein of the present invention can be employed in comparative assays to demonstrate a reduced level of leakiness in comparison to other known gene-editing systems. Additionally, cell-free assays can be used to measure nuclease activity, for example, Cas9 nuclease activity; these methods are further described in, for example, Cox, K. J., et al. Chem Sci, 2019, which is incorporated herein in its entirety. It is specifically contemplated that any method known in the art that measures nuclease activity of a gene-editing protein can be used herein to assess the inhibitor's ability to inhibit nuclease activity of a gene-editing protein.

### Guide RNA

For certain gene-editing systems, e.g., CRISPR-Cas and ZFN, a guide RNA is additionally required. As used herein, the term “guide RNA” generally refers to an RNA molecule (or a group of RNA molecules collectively) that can bind to a CRISPR-associated nuclease, e.g., an endonuclease, for example, a Cas protein, and aid in targeting the endonuclease to a specific location within a target polynucleotide (e.g., a DNA). A guide RNA can comprise a crRNA segment and a tracrRNA segment. As used herein, the term “crRNA” or “crRNA segment” refers to an RNA molecule or portion thereof that includes a polynucleotide-targeting guide sequence, a stem sequence, and, optionally, a 5′-overhang sequence. As used herein, the term “tracrRNA” or “tracrRNA segment” refers to an RNA molecule or portion thereof that includes a protein-binding segment (e.g., the protein-binding segment is capable of interacting with a CRISPR-associated protein, such as a Cas9). The term “guide RNA” encompasses a single guide RNA (sgRNA), where the crRNA segment and the tracrRNA segment are located in the same RNA molecule. The term “guide RNA” also encompasses, collectively, a group of two or more RNA molecules, where the crRNA segment and the tracrRNA segment are located in separate RNA molecules.

A synthetic guide RNA that has “gRNA functionality” is one that has one or more of the functions of naturally occurring guide RNA, such as associating with an endonuclease, or a function performed by the guide RNA in association with an endonuclease. In certain embodiments, the functionality includes binding a target polynucleotide. In certain embodiments, the functionality includes targeting the endonuclease or a gRNA: endonuclease complex to a target polynucleotide. In certain embodiments, the functionality includes nicking a target polynucleotide. In certain embodiments, the functionality includes cleaving a target polynucleotide. In certain embodiments, the functionality includes associating with or binding to the endonuclease. In certain embodiments, the functionality is any other known function of a guide RNA in a CRISPR-associated nuclease system with an endonuclease, including an artificial CRISPR-associated nuclease system with an engineered endonuclease, for example, an engineered Cas protein. In certain embodiments, the functionality is any other function of natural guide RNA. The synthetic guide RNA may have gRNA functionality to a greater or lesser extent than a naturally occurring guide RNA. In certain embodiments, a synthetic guide RNA may have greater functionality as to one property and lesser functionality as to another property in comparison to a similar naturally occurring guide RNA.

Guide RNAs, e.g., for use with the system described herein are known in the art and are further described in U.S. Pat. No. 9,834,791; and Patent Application No. US2013,0254304. Guide RNAs, e.g., for use with a ZFN system are known in the art and are further described in and International Patent Application No. WO2014,186,585. Patents cited herein are incorporated herein by reference in their entirety.

Guide RNA sequences can be readily generated for a given target sequence using prediction software, for example, CRISPRdirect (available on the world wide web at http://crispr.dbels.jp/), see Natio, et al. Bioinformatics. (2015) April 1; 31(7): 1120-1123; ATUM gRNA Design Tool (available on the world wide web at www.atum.bio:ecommerce/cas9/input); an CRISPR-ERA (available on the world wide web at http://crispr-era.stanford.eduu/indexjsp), see Liu, et al. Bioinformatics, (2015) November 15; 31(22): 3676-3678. All references cited herein are incorporated herein by reference in their entireties. Non-limiting examples of publically available gRNA design software include; sgRNA Scorer 1.0, Quilt Universal guide RNA designer, Cas-OFFinder & Cas-Designer, CRISPR-ERA, CRISPR/Cas9 target online predictor, Off-Spotter—for designing gRNAs, CRISPR MultiTargeter, ZiFiT Targeter, CRISPRdirect, CRISPR design from crispr.mit.edu/, E-CRISP etc.

A guide RNA described herein can be modified, e.g., chemically modified. Exemplary chemical modifications of a guide RNA are described in, for example, Patent Application WO2016 089,433, which is incorporated herein by reference in its entirety.

When a nucleic acid encoding one or more single-guide RNAs and a nucleic acid a CRISPR associated nuclease (RNA-guided nuclease) described herein each need to be administered in vivo, the use of an adenovirus associated vector (AAV) is specifically contemplated. Other vectors for simultaneously delivering nucleic acids to all components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector (viral or non-viral) as known in the art or as described herein.

In one embodiment, a transgene can also be present with one part of the CRISPR system, or in a second vector to be co-transfected. The transgene can be part of any CRISPR system used to precisely add the transgene.

### Methods for Identifying Inhibitors of Gene-Editing Proteins

Methods for identifying peptides that inhibit a gene-editing protein are provided herein. In one aspect, a peptide that inhibits a gene-editing protein is identified using a phage display screen. The method comprises (a) immobilizing a gene-editing protein sequence in a well of a microtiter plate; (b) introducing the phage display library to the microtiter plate for a time sufficient to allow for binding of the phage library to the gene-editing protein; (c) identifying any peptide bound to the gene-editing protein as a candidate peptide; and (e) testing/evaluating the candidate peptides identified in steps (a)-(c) through one or more biological assays for their ability to modulate the nuclease activity of a gene-editing protein.

Bacteriophage (phage) display is a technique by which variant polypeptides are displayed as fusion proteins to the coat protein on the surface of bacteriophage particles. Phage display libraries of comprise selectively randomized protein variants (or randomly cloned cDNAs) that can be rapidly and efficiently sorted for those sequences that bind to a target molecule (e.g., a gene-editing gene) with high affinity. Phage display peptide libraries can be used to screen thousands of polypeptides for ones with specific binding properties (e.g., bind to a gene-editing protein). Phage display is further reviewed in, for example, Cwirla, S. E. et al. (1990) Proc. Natl. Acad. Sci. USA; Scott, J. K. and Smith, G. P. (1990) Science; and Smith, G. P. (1991) Current Opin. Biotechnol.; the contents of which are incorporated herein by reference in their entireties.

Methods for using and improving phage display library screens are further described in, e.g., U.S. Application numbers US20070111282A1; US20020150914A1; US20060035223A1; and U.S. Pat. Nos. 5,824,520A; 5,821,047A; 6,506,566B2; 9,062,305B2; 8,372,954B2; 7,893,007B2; 8,685,893B2; the contents of which are incorporated herein by reference in their entireties.

In various embodiments, the gene-editing protein sequence immobilized to the microtiter plate is a full-length sequence of the gene-editing protein, or a fragment of the sequence of the gene-editing protein.

In one embodiment, the gene-editing protein sequence immobilized to the microtiter plate is a Cas protein sequence. In another embodiment, the gene-editing protein sequence immobilized to the microtiter plate is a fragment of the Cas protein sequence. In yet another embodiment, the gene-editing protein sequence immobilized to the microtiter plate is a sequence comprising, or consisting essentially of the HNH domain sequence of the Cas protein.

Another aspect herein provides a method of identifying a peptide that inhibits a gene-editing protein via in silico screening. The method comprises (a) screening peptide libraries using in silico high throughput docking for candidate peptides that are selectively identified for their ability to target and disrupt the nuclease activity of a gene-editing protein; and (b) testing/evaluating the candidate peptides identified in step (a) through one or more biological assays for their ability to modulate the nuclease activity of a gene-editing protein.

In silico screening approaches utilize computer modeling to identify molecules with a high likelihood of binding a given target. In some embodiments, the in silico screen is performed via analyzing a gene-editing protein of interest via crystallography date and defining domains, for example, protein binding domains of the gene-editing protein. The protein binding domains can be expressed as one or more pharmacophore features and/or compiled in a pharmacophore model comprising one or more pharmacophore features. Pharmacophore generation can be according to software designed for such a task. Candidate peptides or molecules (from, for example, one or more chemical libraries) are selected from those peptides or molecules which align to the pharmacophore models. Preferably, candidate molecules are docked and scored in silico for interaction with the given gene-editing protein. Docking and scoring can be according to software known in the art designed for such task. After selection of molecules aligning to one or more pharmacophore models, where such molecules were optionally docked and scored in silico, the selected molecules are obtained, for example by chemical synthesis or from a commercial source. The selected molecules can be measured for binding affinity and/or effect on function (e.g., nuclease activity) for the given gene editing protein.

In silico screening methods are further described in, e.g., U.S. Application numbers US20110071142A1; US20100120754A1; US20080015194A1; and U.S. Pat. No. 7,904,249B2; U.S. Pat. No. 7,058,515B1; the contents of which are incorporated herein by reference in their entireties.

A number of biological assays known in the art can be used to test or evaluate the candidate peptides ability to modulate the nuclease activity of a gene-editing protein identified via a screening method described herein. As one example, the amount of nuclease activity can be determined by employing whole genome sequencing in a cell expressing the gene-editing protein targeted in the screen and the candidate peptide, and a cell expressing only the gene-editing protein. If the candidate peptide inhibits nuclease activity, one would expect to find a lower incidence of mutations (e.g., on-target and off-target) in a cell expressing the peptide. Further, assays for broadly detecting nuclease activity are known in the art. For example, a CRISPRuTest™ Functional Cas9 Activity Assay can be used to measure Cas9 nuclease activity in any mammalian cell system. The CRISPRuTest Kit provides a FACS-based assay to assess the Cas9 nuclease activity in cells expressing Streptococcus pyogenes Cas9. This kit contains pseudoviral packaged lentiviral constructs with a green fluorescent protein (GFP) gene whose fluorescence is disrupted in cells with active Cas9 nuclease activity. Additionally, cell-free assays have been developed to measure Cas nuclease activity. Cell-free assays for measuring Cas nuclease activity is further described in, for example, Cox, K. J., et al. Chem Sci, 2019, which is incorporated herein in its entirety. It is specifically contemplated that any method known in the art that measures nuclease activity of a gene-editing protein can be used herein to assess the peptide's ability to inhibit nuclease activity of a gene-editing protein.

Further, provided herein is a peptide that inhibits a gene-editing protein identified via screening methods described herein. In various embodiment, the inhibitor of the gene-editing protein is any of the peptides that inhibits a gene-editing protein described herein. For example, the peptides that inhibits a gene-editing protein can be constitutively expressed in a cell line described herein, or expressed in a host system described herein.

All publications, patent applications, patents, patent publications and other references cited herein are incorporated by reference in their entireties for the teachings relevant to the sentence and/or paragraph in which the reference is presented.

The examples, which follow, are set forth to illustrate the present invention, and are not to be construed as limiting thereof.

The invention provided herein can further be described in the following numbered paragraphs:
1. A method of manufacturing vectors containing a heterologous gene-editing protein, the method comprising, providing
   (a) transforming a host system with a nucleic acid cassette containing a promoter operably linked to a gene encoding a gene-editing protein, wherein the host system also contains a heterologous inhibitor for the gene-editing protein;
   (b) incubating the host system for a time sufficient for vector production and to release the recombinant vector; and
   (c) recovering the recombinant vector.
2. The method of paragraph 1, wherein the host system is a host cell.
3. The method of any of the preceding paragraphs, wherein the host cell is a mammalian cell or an insect cell.
4. The method of any of the preceding paragraphs, wherein the host system is a cell-free system.
5. The method of any of the preceding paragraphs, wherein the vector is a viral vector.
6. The method of any of the preceding paragraphs, wherein the viral vector is an adeno associated virus (AAV), a lentivirus (LV), a herpes simplex virus (HSV), an adeno virus (AV), or a pox virus (PV).
7. The method of any of the preceding paragraphs, wherein the vector is a DNA or RNA virus.
8. The method of any of the preceding paragraphs, wherein the nucleic acid cassette is flanked by terminal repeats.
9. The method of any of the preceding paragraphs, wherein the viral vector is an AAV vector and the nucleic acid cassette is flanked by inverted terminal repeats (ITRs).
10. The method of any of the preceding paragraphs, wherein the viral vector is an AV vector and the nucleic acid cassette is flanked by inverted terminal repeats (ITRs).
11. The method of any of the preceding paragraphs, wherein the viral vector is an LV vector and the nucleic acid cassette is flanked by long terminal repeats (LTRs).
12. The method of any of the preceding paragraphs, wherein the vector is a Self-Inactivating (SIN) system vector.
13. The method of any of the preceding paragraphs, wherein the gene-editing protein is a Cas protein.
14. The method of any of the preceding paragraphs, wherein the heterologous inhibitor of a gene-editing protein is an anti-CRISPR protein.
15. The method of any of the preceding paragraphs, wherein the heterologous inhibitor of a gene-editing protein is an antibody that binds to the HNH domain of a Cas protein.
16. The method of any of the preceding paragraphs, wherein the antibody is a single chain antibody, a fragment antigen-binding antibody, or an intrabody.
17. The method of any of the preceding paragraphs, wherein binding the HNH domain results in a conformational change to the structure of the Cas protein.
18. The method of any of the preceding paragraphs, wherein binding the HNH domain inactivates the activity of the Cas protein.
19. The method of any of the preceding paragraphs, wherein a second nucleic acid cassette containing a promoter operably linked to a gene encoding the heterologous inhibitor of a gene-editing protein is administered to the host system prior to step (a) of paragraph 1, or co-administered with step (a) of paragraph 1.
20. The method of any of the preceding paragraphs, wherein the host system constitutively expresses the heterologous inhibitor of a gene-editing protein.
21. The method of any of the preceding paragraphs, wherein the host system transiently expresses the heterologous inhibitor of a gene-editing protein.
22. The method of any of the preceding paragraphs, wherein the second nucleic acid cassette is administered by a plasmid, a virus, a liposome, a microcapsule, a non-viral vector, or as naked DNA.
23. A cell line for expressing vectors containing a gene-editing protein with an heterologous inhibitor of the gene-editing protein to prevent leaky expression of the gene-editing protein, the cell line comprising constitutive expression of an inhibitor of a gene-editing protein.
24. The cell line of any of the preceding paragraphs, wherein the cell is a eukaryotic cell or a prokaryotic cell.
25. The cell line of any of the preceding paragraphs, wherein the gene-editing protein is a Cas protein.
26. The method of any of the preceding paragraphs, wherein the heterologous inhibitor of a gene-editing protein is an anti-CRISPR protein.
27. The cell line of any of the preceding paragraphs, wherein the heterologous inhibitor of a gene-editing protein is an antibody that binds to the HNH domain of a Cas protein.
28. The cell line of any of the preceding paragraphs, wherein the antibody is a single chain antibody, a fragment antigen-binding antibody, or an intrabody.
29. The cell line of any of the preceding paragraphs, wherein binding the HNH domain results in a conformational change to the structure of the Cas protein.
30. The cell line of any of the preceding paragraphs, wherein binding the HNH domain inactivates the activity of the Cas protein.
31. The cell line of any of the preceding paragraphs, wherein the gene-editing protein is the CRISPR-Cas 9 gene-editing system.
32. The cell line of any of the preceding paragraphs, wherein the Cas protein is selected from the group consisting of: Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.
33. The cell line of any of the preceding paragraphs, wherein the Cas protein is Cas9.
34. The cell line of any of the preceding paragraphs, wherein the Cas protein is a Cas9 variant selected from Staphylococcus aureus (SaCas9), Streptococcus thermophilus (StCas9), Neisseria meningitidis (NmCas9), Francisella novicida (FnCas9), and Campylobacter jejuni (CjCas9).
35. The cell line of any of the preceding paragraphs, wherein the Cas protein has been modified for gene-editing without double strand DNA breaks (such as CRISPRi or CRISPRa) and is selected from the group consisting of dCas, nCas, and Cas 13.
36. The cell line of any of the preceding paragraphs, wherein the Cas protein is codon optimized for expression in the eukaryotic cell.
37. The cell line of any of the preceding paragraphs, wherein the cell further expresses a gene-editing protein.
38. The cell line of any of the preceding paragraphs, wherein the expression of the gene-editing protein is transient.
39. The cell line of any of the preceding paragraphs, wherein the expression of the gene-editing protein is constitutive.
40. An antibody that binds to the HNH domain of a Cas protein.
41. The antibody of any of the preceding paragraphs, wherein the Cas protein is selected from the group consisting of: Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.
42. The antibody of any of the preceding paragraphs, wherein the Cas protein is a Cas9 variant selected from Staphylococcus aureus (SaCas9), Streptococcus thermophilus (StCas9), Neisseria meningitidis (NmCas9), Francisella novicida (FnCas9), and Campylobacter jejuni (CjCas9).
43. The antibody of any of the preceding paragraphs, wherein the Cas protein has been modified for gene-editing without double strand DNA breaks (such as CRISPRi or CRISPRa) and is selected from the group consisting of dCas, nCas, and Cas 13.
44. The antibody of any of the preceding paragraphs, wherein the antibody is a single chain antibody, a fragment antigen-binding antibody, or an intrabody.
45. The antibody of any of the preceding paragraphs, wherein binding the HNH domain results in a conformational change to the structure of the Cas protein.
46. The antibody of any of the preceding paragraphs, wherein binding the HNH domain inactivates the activity of the Cas protein.
47. The antibody of any of the preceding paragraphs, wherein at least 50%, 60%, 70%, 80%, 90%,
99% or more of the activity is inactivated.
48. The method of any of the preceding paragraphs, wherein the gene-editing protein is the CRISPR-Cas 9 gene-editing system.
49. The method of any of the preceding paragraphs, wherein the Cas protein is selected from the group consisting of: Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.
50. The method of any of the preceding paragraphs, wherein the Cas protein is Cas9.
51. The method of any of the preceding paragraphs, wherein the Cas protein is a Cas9 variant selected from Staphylococcus aureus (SaCas9), Streptococcus thermophilus (StCas9), Neisseria meningitidis (NmCas9), Francisella novicida (FnCas9), and Campylobacter jejuni (CjCas9).
52. The method of any of the preceding paragraphs, wherein the Cas protein has been modified for gene-editing without double strand DNA breaks (such as CRISPRi or CRISPRa) and is selected from the group consisting of dCas, nCas, and Cas 13.
53. The method of any of the preceding paragraphs, wherein the Cas protein is codon optimized for expression in the eukaryotic cell.
54. The method of any of the preceding paragraphs 1, wherein the cell further expresses a gene-editing protein.
55. The method of any of the preceding paragraphs, wherein the expression of the gene-editing protein is transient.
56. The method of any of the preceding paragraphs, wherein the expression of the gene-editing protein is constitutive.
57. The method of any of the preceding paragraphs, wherein binding the HNH domain inactivates the activity of the Cas protein.
58. The method of any of the preceding paragraphs, wherein at least 50%, 60%, 70%, 80%, 90%,
99% or more of the activity is inactivated.
59. A method of identifying a peptide capable of inhibiting a gene-editing protein, the method comprising
   (a) immobilizing a gene-editing protein sequence in a well of a microtiter plate;
   (b) introducing the phage display library to the microtiter plate for a time sufficient to allow for binding of the phage to the gene-editing protein;
   (c) identifying any peptide bound to the gene-editing protein as a candidate peptide; and
   (d) testing/evaluating the candidate peptides identified in steps (a)-(c) through one or more in vitro assays for their ability to modulate the nuclease activity of a gene-editing protein.
60. The method of any of the preceding paragraphs, wherein the gene-editing protein sequence is a Cas protein sequence.
61. The method of any of the preceding paragraphs, wherein the gene-editing protein sequence is a HNH domain sequence of a Cas protein.
62. The method of any of the preceding paragraphs, wherein the phage display library is a phage display peptide library.
63. A method of identifying a peptide capable of inhibiting a gene-editing protein, the method comprising the steps of:
   (a) screening peptide libraries using in silico high throughput docking for candidate peptides that are selectively identified for their ability to target and disrupt the nuclease activity of a gene-editing protein; and
   (b) testing/evaluating the candidate peptides identified in step (a) through one or more in vitro assays for their ability to modulate the nuclease activity of a gene-editing protein.
64. A peptide that inhibits a gene-editing protein identified using the methods of any of the preceding paragraphs.
65. The method of any of the preceding paragraphs, wherein the inhibitor of the gene-editing protein is the peptide of paragraph 64.
66. The cell line of any of the preceding paragraphs, wherein the inhibitor of the gene-editing protein is the peptide of paragraph 64.
67. The method of any preceding paragraph, wherein the promoter is an inducible promoter.
68. The method of any preceding paragraph, wherein the promoter is a tissue-specific promoter.

### EXAMPLES

### Example 1: Manufacturing of Viral Vectors Using Cells Having Constitutive Expression of an Inhibitor of a Gene-Editing Protein

Described herein in is a method of manufacturing viral vectors from Pro10/HEK293 cells that have been engineered to stably express an inhibitor of a gene-editing protein.

The stable cell line, Pro10/HEK293, as described in U.S. Pat. No. 9,441,206, is ideal for scalable production of AAV vectors. This cell line can be contacted with an expression vector used to express the amino acid sequence of the anti-CRISPR protein, AcrIIC1 (SEQ ID NO: 6). Clonal populations having ArcIIC1 expression integrated into their genome are selected using methods well known in the art. Expression of ArcIIC1 protein is confirmed via western-blotting.

Pro10/HEK293 cells stably expressing AcrIIC1 are transfected with the pAAV-Self-Cas9 plasmid (i.e., containing the self-inactivating CRISPR/Cas system), and a Packaging plasmid encoding Rep2 and serotype-specific Cap2: AAV-Rep/Cap, and the Ad-Helper plasmid (XX680: encoding adenoviral helper sequences). Cells expressing these constructs will produce mature AAV capsids containing the Self-Inactivating/Deleting AAV-CRISPR System and ArcIIC1 for in vivo genome editing.

Transfection. On the day of transfection, the cells are counted using a ViCell XR Viability Analyzer (Beckman Coulter) and diluted for transfection. To mix the transfection cocktail the following reagents are added to a conical tube in this order: plasmid DNA, OPTIMEM® I (Gibco) or OptiPro SFM (Gibco), or other serum free compatible transfection media, and then the transfection reagent at a specific ratio to plasmid DNA. The cocktail is inverted to mix prior to being incubated at room temperature. The transfection cocktail is pipetted into the flasks and placed back in the shaker/incubator. All optimization studies are carried out at 30 mL culture volumes followed by validation at larger culture volumes. Cells are harvested 48 hours post-transfection.

Production of rAAV Using Wave Bioreactor Systems. Wave bags are seeded 2 days prior to transfection. Two days post-seeding the wave bag, cell culture counts are taken and the cell culture is then expanded/diluted before so transfection. The wave bioreactor cell culture is then transfected. Cell culture is harvested from the wave bio-reactor bag at least 48 hours post-transfection.

Titer: AAV titers are calculated after DNase digestion using qPCR against a standard curve (AAV ITR specific) and primers specific to the Self-Deleting AAV-CRISPR System (Cas9).

Harvesting Suspension Cells from Shaker Flasks and 60 Wave Bioreactor Bags. 48 hours post-transfection, cell cultures are collected into 500 mL polypropylene conical tubes (Corning) either by pouring from shaker flasks or pumping from wave bioreactor bags. The cell culture is then centrifuged at 655×g for 10 min using a Sorvall RC3C plus centrifuge and H6000A rotor. The supernatant is discarded, and the cells are resuspended in 1×PBS, transferred to a 50 mL conical tube, and centrifuged at 655×g for 10 mM. At this point, the pellet can either be stored in NLT-60° C. or continued through purification.

Titering rAAV from Cell Lysate Using qPCR. 10 mL of cell culture is removed and centrifuged at 655×g for 10 min using a Sorvall RC3C plus centrifuge and H6000A rotor. The supernatant is decanted from the cell pellet. The cell pellet is then resuspended in 5 mL of DNase buffer (5 mM CaCl2, 5 mM MgCl2, 50 mM Tris-HCl pH 8.0) followed by sonication to lyse the cells efficiently. 300 uL is then removed and placed into a 1.5 mL microfuge tube. 140 units of DNase I is then added to each sample and incubated at 37° C. for 1 hour. To determine the effectiveness of the DNase digestion, 4-5 mg of AcrIIC1 plasmid is spiked into a non-transfected cell lysate with is and without the addition of DNase. 50 μL of EDTA/Sarkosyl solution (6.3% sarkosyl, 62.5 mM EDTA pH 8.0) is added to each tube and incubated at 70° C. for 20 minutes. 50 μL of Proteinase K (10 mg/mL) is then added and incubated at 55° C. for at least 2 hours. Samples are boiled for 15 minutes to inactivate the Proteinase K. An aliquot is removed from each sample to be analyzed by qPCR. Two qPCR reactions are carried out in order to effectively determine how much rAAV vector is generated per cell. One qPCR reaction is set up using a set of primers 2s designed to bind to a homologous sequence on the backbones of plasmids XX680, pXR2 and AcrIIC1. The second qPCR reaction is set up using a set of primers to bind and amplify a region within the AcrIIC1 gene. qPCR is conducted using Sybr green reagents and Light cycler 480 from 30 Roche. Samples are denatured at 95° C. for 10 minutes followed by 45 cycles (90° C. for 10 sec, 62° C. for 10 sec and 72° C. for 10 sec) and melting curve (1 cycle 99° C. for 30 sec, 65° C. for 1 minute continuous).

Purification of rAAV from Crude Lysate. Each cell pellet is adjusted to a final volume of 10 mL. The pellets are vortexed briefly and sonicated for 4 minutes at 30% yield in one second on, one second off bursts. After sonication, 550 U of DNase is added and incubated at 37° C. for 45 minutes. The pellets are then centrifuged at 9400×g using the Sorvall RCSB centrifuge and HS-4 rotor to pellet the cell debris and the clarified lysate is transferred to a Type70Ti centrifuge tube (Beckman 361625). In regard to harvesting and lysing the suspension HEK293 cells for isolation of rAAV, one skilled in the art can use as mechanical methods such as microfluidization or chemical methods such as detergents, etc., followed by a clarification step using depth filtration or Tangential Flow Filtration (TFF).

AAV Vector Purification. Clarified AAV lysate is purified by column chromatography methods as one skilled in the art would be aware of and described in the following manuscripts (Allay et al., Davidoff et al., Kaludov et al., Zolotukhin et al., Zolotukin et al, etc), which are incorporated herein by reference in their entireties.

### Example 2: Manufacturing of Viral Vectors Using Cells Having Transient Expression of an Inhibitor of a Gene-Editing Protein

Described herein is the triple-transfection strategy for anti-Self-Inactivating/Deleting AAV-CRISPR System Production (FIG. 1A-1D). Using this method, rAAV vectors are generated using the following steps: production, purification, and titration.

Production: Transfections were performed in HEK293 cells using the pAAV-Self-Cas9 plasmid (the AAV-Genome plasmid contains the self-inactivating CRISPR/Cas system), a Packaging plasmid (encoding Rep2 and serotype-specific Cap2: AAV-Rep/Cap), and the Ad-Helper plasmid (XX680: encoding adenoviral helper sequences) or anti-CRISPR-Ad-Helper plasmid (constructed based on plasmid Ad-Helper plasmid, contains the anti-Self-Inactivating/Deleting CRISPR System (FIG. 2), which produces mature AAV capsids containing the Self-Inactivating/Deleting AAV-CRISPR System for in vivo genome editing.

Purification: At harvest, the transfected cell culture was lysed with detergent to liberate AAV particles. AAV preparations were purified by affinity chromatography, gradient ultracentrifugation and ultrafiltration.

Titer: AAV titers were calculated after DNase digestion using qPCR against a standard curve (AAV ITR specific) and primers specific to the Self-Deleting AAV-CRISPR System (Cas9).

Next-Generation Sequencing (NGS) was used to sequence all the DNA packaged inside the AAV particles and determine the identity of the packaged DNA. Additionally, NGS results are aligned with the entire self-inactivating AAV genome to verify the integrity of the genome elements and confirming insertion and/or deletion mutations. Western Blot analysis was performed to detect the Cas9 protein expression and the effects of the self-inactivating editing system. Western blotting was performed using anti-Cas9 protein and anti-B-Actin (as a loading control) antibodies.

The single vector system allowed for efficient production of Self-inactivating AAV-CRISPR vectors. The mutations derived from the Self-Inactivating Cas System represented less than 5% in rAAV stocks and Cas9 protein is readily detectable by western blotting in HEK293 cells 48 h after AAV-Self-Cas9 transduction, like the negative control experiment (AAV-Self-Cas9 without self-deleting gRNA).

The experiments presented in Example 2 are repeated with anti-CRISPR proteins AcrIIA4 (SEQ ID NO: 7) and AcrIIA2 (SEQ ID NO: 8), and similar results are observed.

Further, the experiments presented in Example 2 are repeated in a cell line expressing an inhibitory antibody that binds within the HNH domain of Cas, Anti-Cas9 Antibody, D10A/H840A Mutant (Millipore Sigma; Burlington, Mass.), and similar results are observed.

### Example 3: Manufacturing of Viral Vectors Using Cells Having Transient Expression of an Inhibitor of a Gene-Editing Protein and a Transgene

Described herein is the triple-transfection strategy for anti-Self-Inactivating/Deleting AAV-CRISPR System Production and expression of the human factor IX minigene. Using this method, rAAV vectors are generated using the following steps: production, purification, and titration.

Production: Transfections are performed in HEK293 cells using the pAAV-Self-Cas9 and factor IX plasmid (the AAV-Genome plasmid contains the self-inactivating CRISPR/Cas system and human factor IX minigene), a Packaging plasmid (encoding Rep2 and serotype-specific Cap2: AAV-Rep/Cap), and the Ad-Helper plasmid (XX680: encoding adenoviral helper sequences) or anti-CRISPR-Ad-Helper plasmid (constructed based on plasmid Ad-Helper plasmid, contains the anti-Self-Inactivating/Deleting CRISPR System, which produces mature AAV capsids containing the Self-Inactivating/Deleting AAV-CRISPR System for in vivo genome editing.

Purification: At harvest, the transfected cell culture is lysed with detergent to liberate AAV particles. AAV preparations are purified by affinity chromatography, gradient ultracentrifugation and ultrafiltration.

Titer: AAV titers are calculated after DNase digestion using qPCR against a standard curve (AAV ITR specific), primers specific to the Self-Deleting AAV-CRISPR System (Cas9) and primers specific to the factor IX minigene.

Next-Generation Sequencing (NGS) is used to sequence all the DNA packaged inside the AAV particles and determine the identity of the packaged DNA. Additionally, NGS results are aligned with the entire self-inactivating AAV genome to verify the integrity of the genome elements and confirming insertion and/or deletion mutations. Western Blot analysis is performed to detect the Cas9 protein expression and the effects of the self-inactivating editing system. Western blotting is performed using anti-Cas9 protein and anti-B-Actin (as a loading control) antibodies.

The experiments presented in Example 3 are repeated with anti-CRISPR proteins AcrIIA4 (SEQ ID NO: 7) and AcrIIA2 (SEQ ID NO: 8), and similar results are observed.

### REFERENCES

1) Lee J. et al., Potent Cas9 Inhibition in Bacterial and Human Cells by AcrIIC4 and AcrIIC5 Anti-CRISPR Proteins. mBio. 2018 November-December; 9(6): e02321-18.
2) Harrington L B et al., A Broad-Spectrum Inhibitor of CRISPR-Cas9. Cell. 2017 Sep. 7; 170(6):1224-1233.e15.
3) Shin et al., Disabling Cas9 by an anti-CRISPR DNA mimic. Science Advances 12 Jul. 2017: Vol. 3, no. 7.
4) Liu et al., Phage AcrIIA2 DNA Mimicry: Structural Basis of the CRISPR and Anti-CRISPR Arms Race. Mol Cell. 2019 Feb. 7; 73(3):611-620.e3.
5) Li C. et al., HDAd5/35++ Adenovirus Vector Expressing Anti-CRISPR Peptides Decreases CRISPR/Cas9 Toxicity in Human Hematopoietic Stem Cells. Mol Ther Methods Clin Dev. 2018 May 1; 9:390-401.
6) Bubeck F. et al., Engineered anti-CRISPR proteins for optogenetic control of CRISPR-Cas9. Nat Methods. 2018 November; 15(11):924-927.

1. A method of manufacturing vectors containing a heterologous gene-editing protein, the method comprising, providing
(a) transforming a host system with a nucleic acid cassette containing a promoter operably linked to a gene encoding a gene-editing protein, wherein the host system also contains a heterologous inhibitor for the gene-editing protein;
(b) incubating the host system for a time sufficient for vector production and to release the recombinant vector; and
(c) recovering the recombinant vector.

2. The method of claim 1, wherein the host system is a host cell.

3. The method of claim 2, wherein the host cell is a mammalian cell or an insect cell.

4. The method of claim 1, wherein the host system is a cell-free system.

5. The method of claims 1-4, wherein the vector is a viral vector.

6. The method of claim 5, wherein the viral vector is an adeno associated virus (AAV), a lentivirus (LV), a herpes simplex virus (HSV), an adeno virus (AV), or a pox virus (PV).

7. The method of claims 1-5, wherein the vector is a DNA or RNA virus.

8. The method of claim 5, or claim 7, wherein the nucleic acid cassette is flanked by terminal repeats.

9. The method of claim 8, wherein the viral vector is an AAV vector and the nucleic acid cassette is flanked by inverted terminal repeats (ITRs).

10. The method of claim 8, wherein the viral vector is an AV vector and the nucleic acid cassette is flanked by inverted terminal repeats (ITRs).

11. The method of claim 8, wherein the viral vector is an LV vector and the nucleic acid cassette is flanked by long terminal repeats (LTRs).

12. The method of claim 1, wherein the vector is a Self-Inactivating (SIN) system vector.

13. The method of claim 2, wherein the gene-editing protein is a Cas protein.

14. The method of any of claims 2-3, wherein the heterologous inhibitor of a gene-editing protein is an anti-CRISPR protein.

15. The method of claim 1, wherein the heterologous inhibitor of a gene-editing protein is an antibody that binds to the HNH domain of a Cas protein.

16. The method of claim 15, wherein the antibody is a single chain antibody, a fragment antigen-binding antibody, or an intrabody.

17. The method of claim 15, wherein binding the HNH domain results in a conformational change to the structure of the Cas protein.

18. The method of claim 15, wherein binding the HNH domain inactivates the activity of the Cas protein.

19. The method of claims 1-18 wherein a second nucleic acid cassette containing a promoter operably linked to a gene encoding the heterologous inhibitor of a gene-editing protein is administered to the host system prior to step (a) of claim 1, or co-administered with step (a) of claim 1.

20. The method of claims 1-19, wherein the host system constitutively expresses the heterologous inhibitor of a gene-editing protein.

21. The method of claims 1-19, wherein the host system transiently expresses the heterologous inhibitor of a gene-editing protein.

22. The method of claim 1, wherein the second nucleic acid cassette is administered by a plasmid, a virus, a liposome, a microcapsule, a non-viral vector, or as naked DNA.

23. A cell line for expressing vectors containing a gene-editing protein with an heterologous inhibitor of the gene-editing protein to prevent leaky expression of the gene-editing protein, the cell line comprising constitutive expression of an inhibitor of a gene-editing protein.

24. The cell line of claim 23, wherein the cell is a eukaryotic cell or a prokaryotic cell.

25. The cell line of claim 24, wherein the gene-editing protein is a Cas protein.

26. The method of any of claims 23-24, wherein the heterologous inhibitor of a gene-editing protein is an anti-CRISPR protein.

27. The cell line of claim 23, wherein the heterologous inhibitor of a gene-editing protein is an antibody that binds to the HNH domain of a Cas protein.

28. The cell line of claim 27, wherein the antibody is a single chain antibody, a fragment antigen-binding antibody, or an intrabody.

29. The cell line of claim 27, wherein binding the HNH domain results in a conformational change to the structure of the Cas protein.

30. The cell line of claim 27, wherein binding the HNH domain inactivates the activity of the Cas protein.

31. The cell line of claim 23, wherein the gene-editing protein is the CRISPR-Cas 9 gene-editing system.

32. The cell line of claims 25 and 27, wherein the Cas protein is selected from the group consisting of: Cpf1, C2c1, C2c3, Cas12a, Cas12b, Cas12c, Cas12d, Cas12e, Cas13a, Cas13b, and Cas13c, Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.

33. The cell line of claims 25 and 27, wherein the Cas protein is Cas9.

34. The cell line of claims 25 and 27, wherein the Cas protein is a Cas9 variant selected from Staphylococcus aureus (SaCas9), Streptococcus thermophilus (StCas9), Neisseria meningitidis (NmCas9), Francisella novicida (FnCas9), and Campylobacter jejuni (CjCas9).

35. The cell line of claims 25 and 27, wherein the Cas protein has been modified for gene-editing without double strand DNA breaks (such as CRISPRi or CRISPRa) and is selected from the group consisting of dCas, nCas, and Cas 13.

36. The cell line of any of claims 32-35, wherein the Cas protein is codon optimized for expression in the eukaryotic cell.

37. The cell line of claim 23, wherein the cell further expresses a gene-editing protein.

38. The cell line of claim 37, wherein the expression of the gene-editing protein is transient.

39. The cell line of claim 37, wherein the expression of the gene-editing protein is constitutive.

40. An antibody that binds to the HNH domain of a Cas protein.

41. The antibody of claim 40, wherein the Cas protein is selected from the group consisting of: Cpf1, C2c1, C2c3, Cas12a, Cas12b, Cas12c, Cas12d, Cas12e, Cas13a, Cas13b, and Cas13c, Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.

42. The antibody of claim 40, wherein the Cas protein is a Cas9 variant selected from Staphylococcus aureus (SaCas9), Streptococcus thermophilus (StCas9), Neisseria meningitidis (NmCas9), Francisella novicida (FnCas9), and Campylobacter jejuni (CjCas9).

43. The antibody of claim 40, wherein the Cas protein has been modified for gene-editing without double strand DNA breaks (such as CRISPRi or CRISPRa) and is selected from the group consisting of dCas, nCas, and Cas 13.

44. The antibody of claim 40, wherein the antibody is a single chain antibody, a fragment antigen-binding antibody, or an intrabody.

45. The antibody of claim 58, wherein binding the HNH domain results in a conformational change to the structure of the Cas protein.

46. The antibody of claim 40, wherein binding the HNH domain inactivates the activity of the Cas protein.

47. The antibody of claim 46, wherein at least 50%, 60%, 70%, 80%, 90%, 99% or more of the activity is inactivated.

48. The method of claims 1-22, wherein the gene-editing protein is the CRISPR-Cas 9 gene-editing system.

49. The method of claims 13 and 15, wherein the Cas protein is selected from the group consisting of: Cpf1, C2c1, C2c3, Cas12a, Cas12b, Cas12c, Cas12d, Cas12e, Cas13a, Cas13b, and Cas13c, Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.

50. The method of claims 13 and 15, wherein the Cas protein is Cas9.

51. The method of claims 13 and 15, wherein the Cas protein is a Cas9 variant selected from Staphylococcus aureus (SaCas9), Streptococcus thermophilus (StCas9), Neisseria meningitidis (NmCas9), Francisella novicida (FnCas9), and Campylobacter jejuni (CjCas9).

52. The method of claims 13 and 15, wherein the Cas protein has been modified for gene-editing without double strand DNA breaks (such as CRISPRi or CRISPRa) and is selected from the group consisting of dCas, nCas, and Cas 13.

53. The method of claims 49-52, wherein the Cas protein is codon optimized for expression in the eukaryotic cell.

54. The method of claim 1, wherein the cell further expresses a gene-editing protein.

55. The method of claim 54, wherein the expression of the gene-editing protein is transient.

56. The method of claim 54, wherein the expression of the gene-editing protein is constitutive.

57. The method of claim 15, wherein binding the HNH domain inactivates the activity of the Cas protein.

58. The method of claim 57, wherein at least 50%, 60%, 70%, 80%, 90%, 99% or more of the activity is inactivated.

59. A method of identifying a peptide capable of inhibiting a gene-editing protein, the method comprising
a. immobilizing a gene-editing protein sequence in a well of a microtiter plate;
b. introducing the phage display library to the microtiter plate for a time sufficient to allow for binding of the phage to the gene-editing protein;
c. identifying any peptide bound to the gene-editing protein as a candidate peptide; and
d. testing/evaluating the candidate peptides identified in steps (a)-(c) through one or more in vitro assays for their ability to modulate the nuclease activity of a gene-editing protein.

60. The method of claim 59, wherein the gene-editing protein sequence is a Cas protein sequence.

61. The method of claim 59, wherein the gene-editing protein sequence is a HNH domain sequence of a Cas protein.

62. The method of claim 59, wherein the phage display library is a phage display peptide library.

63. A method of identifying a peptide capable of inhibiting a gene-editing protein, the method comprising the steps of:
a. screening peptide libraries using in silico high throughput docking for candidate peptides that are selectively identified for their ability to target and disrupt the nuclease activity of a gene-editing protein; and
b. testing/evaluating the candidate peptides identified in step (a) through one or more in vitro assays for their ability to modulate the nuclease activity of a gene-editing protein.

64. A peptide that inhibits a gene-editing protein identified using the methods of claims 59-63.

65. The method of claim 1, wherein the inhibitor of the gene-editing protein is the peptide of claim 64.

66. The cell line of claim 23, wherein the inhibitor of the gene-editing protein is the peptide of claim 64.

67. The method of any preceding claim, wherein the promoter is an inducible promoter.

68. The method of any preceding claim, wherein the promoter is a tissue-specific promoter.


SEQUENCE LISTING (14 sequences)

SEQ ID NO: 1 - protein, 9 aa, Unknown
  Description of Unknown “LAGLIDADG” family peptide motif sequence
LAGLIDADG

SEQ ID NO: 2 - protein, 887 aa, Natronobacterium gregoryi
MTVIDLDSTTTADELTSGHTYDISVTLTGVYDNTDEQHPRMSLAFEQDNGERRYITLWKNTTPKDVFTYDYATGSTYIFTNIDYEVKDGYENLTATYQTTVENATAQEVGTTDEDETFAGGEPLDHHLDDALNETPDDAETESDSGHVMTSFASRDQLPEWTLHTYTLTATDGAKTDTEYARRTLAYTVRQELYTDHDAAPVATDGLMLLTPEPLGETPLDLDCGVRVEADETRTLDYTTAKDRLLARELVEEGLKRSLWDDYLVRGIDEVLSKEPVLTCDEFDLHERYDLSVEVGHSGRAYLHINFRHRFVPKLTLADIDDDNIYPGLRVKTTYRPRRGHIVWGLRDECATDSLNTLGNQSVVAYHRNNQTPINTDLLDAIEAADRRVVETRRQGHGDDAVSFPQELLAVEPNTHQIKQFASDGFHQQARSKTRLSASRCSEKAQAFAERLDPVRLNGSTVEFSSEFFTGNNEQQLRLLYENGESVLTFRDGARGAHPDETFSKGIVNPPESFEVAVVLPEQQADTCKAQWDTMADLLNQAGAPPTRSETVQYDAFSSPESISLNVAGAIDPSEVDAAFVVLPPDQEGFADLASPTETYDELKKALANMGIYSQMAYFDRFRDAKIFYTRNVALGLLAAAGGVAFTTEHAMPGDADMFIGIDVSRSYPEDGASGQINIAATATAVYKDGTILGHSSTRPQLGEKLQSTDVRDIMKNAILGYQQVTGESPTHIVIHRDGFMNEDLDPATEFLNEQGVEYDIVEIRKQPQTRLLAVSDVQYDTPVKSIAAINQNEPRATVATFGAPEYLATRDGGGLPRPIQIERVAGETDIETLTRQVYLLSQSHIQVHNSTARLPITTAYADQASTHATKGYLVQTGAFESNVGFL

SEQ ID NO: 3 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 4 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 5 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 6 - protein, 92 aa, Brackiella oedipodis
MAKEVFKLKPELVTYKGCGWALACIKDGEIIDLTYVRDLGIEEYDENFDGLEPEIIYYDVVASQACKEVAYRYEEMGEFTFGLCSCWEFNVM

SEQ ID NO: 7 - protein, 87 aa, Listeria monocytogenes
MNINDLIREIKNKDYTVKLSGTDSNSITQLIIRVNNDGNEYVISESENESIVEKFISAFKNGWNQEYEDEEEFYNDMQTITLKSELN

SEQ ID NO: 8 - protein, 123 aa, Listeria monocytogenes
MTLTRAQKKYAEAMHEFINMVDDFEESTPDFAKEVLHDSDYVVITKNEKYAVALCSLSTDECEYDTNLYLDEKLVDYSTVDVNGVTYYINIVETNDIDDLEIATDEDEMKSGNQEIILKSELK

SEQ ID NO: 9 - DNA, 199 bp, Artificial Sequence
  Description of Artificial Sequence Synthetic polynucleotide
ggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 10 - DNA, 402 bp, Artificial Sequence
  Description of Artificial Sequence Synthetic polynucleotide
caggctttcactttctcgccaacttacaaggcctttctgtgtaaacaatacctgaacctttaccccgttgcccggcaacggccaggtctgtgccaagtgtttgaggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 11 - DNA, 103 bp, Artificial Sequence
  Description of Artificial Sequence Synthetic polynucleotide
caggctttcactttctcgccaacttacaaggcctttctgtgtaaacaatacctgaacctttaccccgttgcccggcaacggccaggtctgtgccaagtgtttg

SEQ ID NO: 12 - DNA, 212 bp, Artificial Sequence
  Description of Artificial Sequence Synthetic polynucleotide
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccag

SEQ ID NO: 13 - DNA, 305 bp, Artificial Sequence
  Description of Artificial Sequence Synthetic polynucleotide
aggttaatttttaaaaagcagtcaaaagtccaagtggcccttggcagcatttactctctctgtttgctctggttaataatctcaggagcacaaacattcctgtaccggcccgggaggcgccctttggaccttttgcaatcctggcgcactgaacccttgacccctgccctgcagcccccgcagcttgctgtttgcccactctatttgcccagccccagccctggagagtcctttagcagggcaaagtgcaacataggcagaccttaagggatgactcagtaacagataagctttgtgtgcctgca

SEQ ID NO: 14 - DNA, 180 bp, Artificial Sequence
  Description of Artificial Sequence Synthetic polynucleotide
aggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtg